SEQUENCE LISTING (10 sequences)

SEQ ID NO: 1 - DNA, 906 bp, Thermomyces lanuginosus
gcagctagggctgttgtctttataatacgactcactatagggagaccacaagaaggagatatacatatggaggtctcgcaggatctgtttaaccagttcaatctctttgcacagtattctgcagccgcatactgcggaaaaaacaatgatgccccagctggtacaaacattacgtgcacgggaaatgcctgccccgaggtagagaaggcggatgcaacgtttctctactcgtttgaagactctggagtgggcgatgtcaccggcttccttgctctcgacaacacgaacaaattgatcgtcctctctttccgtggctctcgttccatagagaactggatcgggaatcttaacttcgacttgaaagaaataaatgacatttgctccggctgcaggggacatgacggcttcacttcgtcctggaggtctgtagccgatacgttaaggcagaaggtggaggatgctgtgagggagcatcccgactatcgcgtggtgtttaccggacatagcttgggtggtgcattggcaactgttgccggagcagacctgcgtggaaatgggtatgatatcgacgtgttttcatatggcgccccccgagtcggaaacagggcttttgcagaattcctgaccgtacagaccggcggaacactctaccgcattacccacaccaatgatattgtccctagactcccgccgcgcgaattcggttacagccattctagcccagagtactggatcaaatctggaacccttgtccccgtcacccgaaacgatatcgtgaagatagaaggcatcgatgccaccggcggcaataaccagcctaacattccggatatccctgcgcacctatggtacttcgggttaattgggacatgtctttaataagataagtggttcgctccgtttttt

SEQ ID NO: 2 - DNA, 813 bp, Thermomyces lanuginosus
atggaggtctcgcaggatctgtttaaccagttcaatctctttgcacagtattctgcagccgcatactgcggaaaaaacaatgatgccccagctggtacaaacattacgtgcacgggaaatgcctgccccgaggtagagaaggcggatgcaacgtttctctactcgtttgaagactctggagtgggcgatgtcaccggcttccttgctctcgacaacacgaacaaattgatcgtcctctctttccgtggctctcgttccatagagaactggatcgggaatcttaacttcgacttgaaagaaataaatgacatttgctccggctgcaggggacatgacggcttcacttcgtcctggaggtctgtagccgatacgttaaggcagaaggtggaggatgctgtgagggagcatcccgactatcgcgtggtgtttaccggacatagcttgggtggtgcattggcaactgttgccggagcagacctgcgtggaaatgggtatgatatcgacgtgttttcatatggcgccccccgagtcggaaacagggcttttgcagaattcctgaccgtacagaccggcggaacactctaccgcattacccacaccaatgatattgtccctagactcccgccgcgcgaattcggttacagccattctagcccagagtactggatcaaatctggaacccttgtccccgtcacccgaaacgatatcgtgaagatagaaggcatcgatgccaccggcggcaataaccagcctaacattccggatatccctgcgcacctatggtacttcgggttaattgggacatgtctttaa

SEQ ID NO: 3 - protein, 270 aa, Thermomyces lanuginosus
MEVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVTRNDIVKIEGIDATGGNNQPNIPDIPAHLWYFGLIGTCL

SEQ ID NO: 4 - DNA, 906 bp, Thermomyces lanuginosus
gcagctagggctgttgtctttataatacgactcactatagggagaccacaagaaggagatatacatatggaggtctcgcaggatctgtttaaccagttcaatctctttgcacagtattctgcagccgcatactgcggaaaaaacaatgatgccccagctggtacaaacattacgtgcacgggaaatgcctgccccgaggtagagaaggcggatgcaacgtttctctactcgtttgaagactctggagtgggcgatgtcaccggcttccttgctctcgacaacacgaacaaattgatcgtcctctctttccgtggctctcgttccatagagaactggatcgggaatcttaacttcgacttgaaagaaataaatgacatttgctccggctgcaggggacatgacggcttcacttcgtcctggaggtctgtagccgatacgttaaggcagaaggtggaggatgctgtgagggagcatcccgactatcgcgtggtgtttaccgggcatgcattgggtggtgcattggcaactgttgccggagcagacctgcgtggaaatgggtatgatatcgacgtgttttcatatggcgccccccgagtcggaaacagggcttttgcagaattcctgaccgtacagaccggcggaacactctaccgcattacccacaccaatgatattgtccctagactcccgccgcgcgaattcggttacagccattctagcccagaatactggatcaaatctggaacccttgtccccgtcacccgaaacgatatcgtgaagatagaaggcatcgatgccaccggcggcaataaccagcctaacattccggatatccctgcgcacctatggtacttcgggttaattgggacatgtctttaataagataagtggttcgctccgtttttt

SEQ ID NO: 5 - DNA, 813 bp, Thermomyces lanuginosus
atggaggtctcgcaggatctgtttaaccagttcaatctctttgcacagtattctgcagccgcatactgcggaaaaaacaatgatgccccagctggtacaaacattacgtgcacgggaaatgcctgccccgaggtagagaaggcggatgcaacgtttctctactcgtttgaagactctggagtgggcgatgtcaccggcttccttgctctcgacaacacgaacaaattgatcgtcctctctttccgtggctctcgttccatagagaactggatcgggaatcttaacttcgacttgaaagaaataaatgacatttgctccggctgcaggggacatgacggcttcacttcgtcctggaggtctgtagccgatacgttaaggcagaaggtggaggatgctgtgagggagcatcccgactatcgcgtggtgtttaccgggcatgcattgggtggtgcattggcaactgttgccggagcagacctgcgtggaaatgggtatgatatcgacgtgttttcatatggcgccccccgagtcggaaacagggcttttgcagaattcctgaccgtacagaccggcggaacactctaccgcattacccacaccaatgatattgtccctagactcccgccgcgcgaattcggttacagccattctagcccagaatactggatcaaatctggaacccttgtccccgtcacccgaaacgatatcgtgaagatagaaggcatcgatgccaccggcggcaataaccagcctaacattccggatatccctgcgcacctatggtacttcgggttaattgggacatgtctttaa

SEQ ID NO: 6 - protein, 270 aa, Thermomyces lanuginosus
MEVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHALGGALATVAGADLRGNGYDIDVFSYGAPRVGNRAFAEFLTVQTGGTLYRITHTNDIVPRLPPREFGYSHSSPEYWIKSGTLVPVTRNDIVKIEGIDATGGNNQPNIPDIPAHLWYFGLIGTCL

SEQ ID NO: 7 - DNA, 24 bp, Artificial Sequence
  Artificial DNA Primer
aaaaaacggagcgaaccacttatc

SEQ ID NO: 8 - DNA, 22 bp, Artificial Sequence
  Artificial DNA Primer
gcagctagggctgttgtcttta

SEQ ID NO: 9 - DNA, 19 bp, Artificial Sequence
  Artificial DNA Primer
gaggtctcgcaggatctgt

SEQ ID NO: 10 - DNA, 18 bp, Artificial Sequence
  Artificial DNA Primer
gctggggcatcattgttt

(19) United States
(12) Patent Application Publication
(10) Pub. No.: US 2018/0355404 A1
(43) Pub. Date: Dec. 13, 2018

(54) METHODS FOR SELECTING ENZYMES HAVING LIPASE ACTIVITY

(71) Applicant: NOVOZYMES A/S, Bagsvaerd (DK)

(72) Inventors: Robert Blazej, San Francisco, CA (US); Nicholas Toriello, San Carlos, CA (US); Charles Emrich, San Francisco, CA (US); Allan Svendsen, Hoersholm (DK); Jesper Brask, Vaerloese (DK); Anne Olsen, Valby (DK); Kathleen Hirano, Dublin, CA (US); Amy Twite, Berkeley, CA (US)

(73) Assignee: NOVOZYMES A/S, Bagsvaerd (DK)

(21) Appl. No.: 15/563,692

(22) PCT Filed: Apr. 7, 2016
(86) PCT No.: PCT/US2016/026469

§ 371 Date: Oct. 2, 2017

Related U.S. Application Data
(60) Provisional application No. 62/144,001, filed on Apr. 7, 2015.

(51) Int. Cl. C12Q 1/44

(52) U.S. Cl. CPC C12Q 1/44

(57) ABSTRACT

Provided herein are methods and means for enhancing lipase activity. The system makes use of an emulsion for in vitro compartmentalization of a library of synthetic compounds which have a polynucleotide linked to a lipase substrate (e.g., a triglyceride). Expressed polypeptides having greater lipase activity will preferentially hydrolyze the substrate from the linked polynucleotide. Genes encoding polypeptides having less lipase activity will remain linked to the substrate and may be removed to enrich the library for more active variants. Also described are synthetic compounds and emulsions which can be used in the methods.

### CROSS-REFERENCE TO RELATED APPLICATIONS

This application claims the priority benefit of U.S. Provisional Application No. 62/144,001, filed Apr. 7, 2015, the content of which is fully incorporated herein by reference.

### REFERENCE TO A SEQUENCE LISTING

This application contains a Sequence Listing in computer readable form, which is incorporated herein by reference.

### FIELD OF THE INVENTION

The present invention is in the technical field of protein engineering design and selection. More particularly, the present invention relates to enzyme enhancement by means of directed evolution.

### BACKGROUND

Lipases are used for a variety of industrial applications, including, inter alia, household care in detergent compositions for improved stain removal, and baking as a dough stabilizing emulsifier. While lipases have been successfully modified to improve desired properties (See, e.g., WO 2003/060112, WO 2008/079685, and WO 2013/149858) development of lipase variants typically includes protein engineering techniques, such as rational design and/or directed evolution, followed by laborious enzymatic assays to test for improved function. Thus, there is a strong need for methods of rapid and efficient identification of those synthetic genes that encode polypeptides having improved lipase activity.

### SUMMARY

Described herein are systems and components thereof for improving lipase activity. Accordingly, in one aspect is a method of selecting for a polypeptide having lipase activity, the method comprising:
(i) suspending a plurality of synthetic compounds in an aqueous phase, wherein the synthetic compounds individually comprise:
   (a) a polynucleotide encoding for a polypeptide, and
   (b) a lipase substrate linked to said polynucleotide; and
wherein the aqueous phase comprises components for expression of the polypeptide;
(ii) forming a water-in-oil emulsion with the aqueous phase, wherein the synthetic compounds are compartmentalized in aqueous droplets of the emulsion;
(iii) expressing the polypeptides within the aqueous droplets of the emulsion, wherein a polypeptide with lipase activity in an aqueous droplet hydrolyzes one or more synthetic compounds in that droplet; and
(iv) separating the synthetic compounds to recover hydrolyzed and/or non-hydrolyzed synthetic compounds.

In another aspect is a synthetic compound, comprising: (a) a polynucleotide encoding for a polypeptide, and (b) a lipase substrate linked to said polynucleotide.

In another aspect is a method of making the synthetic compound, comprising: (i) linking a lipase substrate to a polynucleotide encoding for a polypeptide, and (ii) recovering the synthetic compound.

In another aspect is a polynucleotide library, comprising a plurality of the synthetic compounds.

In another aspect is a water-in-oil emulsion, comprising the polynucleotide library, wherein the synthetic compounds of the library are compartmentalized in aqueous droplets of the emulsion.

In another aspect is a method of making the emulsion, comprising: (i) suspending the plurality of synthetic compounds in the aqueous phase, and (ii) mixing the suspension of (i) with an oil.

### BRIEF DESCRIPTION OF THE FIGURES

FIG. 1 shows an exemplary diagrammatic representation of the process steps involved in lipase selection in accordance with a negative selection method of the present invention.

FIG. 2 shows the DNA coding sequence (SEQ ID NO: 2) and the deduced amino acid sequence (SEQ ID NO: 3) of the wild-type Thermomyces lanuginosus lipase. SEQ ID NO: 1 (not shown) also contains the 5′ and 3′ nucleotides used for in vitro transcription/translation and PCR amplification (see Examples).

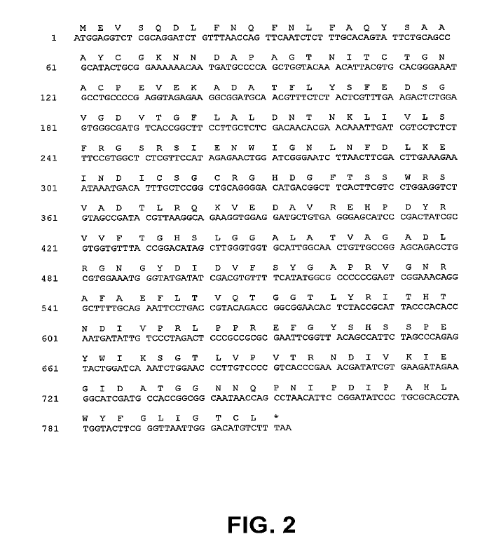

FIG. 3 shows the DNA coding sequence (SEQ ID NO: 5) and the deduced amino acid sequence (SEQ ID NO: 6) of the catalytically inactive Thermomyces lanuginosus lipase. SEQ ID NO: 4 (not shown) also contains the 5′ and 3′ nucleotides used for in vitro transcription/translation and PCR amplification (see Examples).

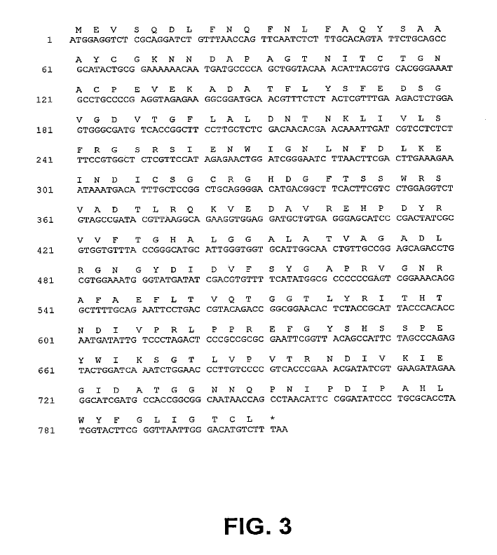

FIG. 4 shows a graphical representation of the self-enrichment of wild-type Thermomyces lanuginosus lipase compared to catalytically inactive Thermomyces lanuginosus lipase by using the systems in the present invention.

FIG. 5 shows an exemplary diagrammatic representation of the process steps involved in lipase selection in accordance with a positive selection method of the present invention.

FIG. 6 shows a graphical representation of the enrichment of synthetic compounds after hydrolysis with wild-type Thermomyces lanuginosus lipase by using a positive selection system of the present invention.

### Definitions

Alkyl: The term “alkyl” means, unless otherwise state, a straight (i.e. unbranched) or branched carbon chain, or combination thereof, which may be fully saturated, mono- or polyunsaturated, and can be optionally substituted with one or more mono-, di-, or multivalent radicals.

Amino acid: The terms “amino acid” or “amino acid residue,” include naturally occurring L-amino acids or residues, unless otherwise specifically indicated. The terms “amino acid” and “amino acid residue” also include D-amino acids as well as chemically modified amino acids, such as amino acid analogs, naturally occurring amino acids that are not usually incorporated into proteins, and chemically synthesized compounds having the characteristic properties of amino acids (collectively, “atypical” amino acids). For example, analogs or mimetics of phenylalanine or proline, which allow the same conformational restriction of the peptide compounds as natural Phe or Pro are included within the definition of “amino acid.”

Coding sequence: The term “coding sequence” or “coding region” means a polynucleotide sequence, which specifies the amino acid sequence of a polypeptide. The boundaries of the coding sequence are generally determined by an open reading frame, which usually begins with the ATG start codon or alternative start codons such as GTG and TTG and ends with a stop codon such as TAA, TAG, and TGA. The coding sequence may be a sequence of genomic DNA, cDNA, a synthetic polynucleotide, and/or a recombinant polynucleotide.

Control sequence: The term “control sequence” means a nucleic acid sequence necessary for polypeptide expression. Control sequences may be native or foreign to the polynucleotide encoding the polypeptide, and native or foreign to each other. Such control sequences include, but are not limited to, a leader sequence, polyadenylation sequence, propeptide sequence, promoter sequence, signal peptide sequence, and transcription terminator sequence. The control sequences may be provided with linkers for the purpose of introducing specific restriction sites facilitating ligation of the control sequences with the coding region of the polynucleotide encoding a polypeptide.

Expression: The term “expression” includes the process of producing a polypeptide from a coding sequence, and may include but is not limited to, transcription, post-transcriptional modification, translation, post-translational modification, and secretion. Expression can be measured—for example, to detect increased expression—by techniques known in the art, such as measuring levels of mRNA and/or translated polypeptide. Expression, as used herein, includes in vitro transcription/translation.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, and the like with a nucleic acid construct or expression vector comprising a polynucleotide described herein (e.g., a polynucleotide encoding a lipase or lipase variant). The term “host cell” encompasses any progeny of a parent cell that is not identical to the parent cell due to mutations that occur during replication.

Linker: The term “linker” or “linked”, as used herein, refers to the chemical attachment of one referenced compound to another referenced compound.

Lipase: The term “lipase” or “lipolytic enzyme” is an enzyme having hydrolytic activity in class EC 3.1,1 as defined by Enzyme Nomenclature. The lipase may have triacylglycerol lipase activity (EC 3.1.1.3), cutinase activity (EC 3.1.1.74), sterol esterase activity (EC 3.1.1.13) and/or wax-ester hydrolase activity (EC 3.1.1.50). In some aspects, the lipase is a triacylglycerol lipase of EC 3.1.1.3. Lipase activity may be determined using methods known in the art (e.g., See EP 14167933.2, filed May 12, 2014).

Mutant: The term “mutant” means a polynucleotide encoding a variant.

Nucleic acid construct: The term “nucleic acid construct” means a nucleic acid molecule, either single- or double-stranded, which comprises one or more control sequences. The construct may be isolated from a naturally occurring gene, modified to contain segments of nucleic acids in a manner that would not otherwise exist in nature, or synthetic.

Operably linked: The term “operably linked” means a configuration in which a control sequence is placed at an appropriate position relative to the coding sequence of a polynucleotide such that the control sequence directs expression of the coding sequence.

Parent or parent lipase: The term “parent” or “parent lipase” means a lipase to which an alteration is made to produce an enzyme variant. The parent may be a naturally occurring (wild-type) polypeptide or a variant or fragment thereof.

Polynucleotide: The term “polynucleotide” refers to a deoxyribonucleotide or ribonucleotide polymer, and unless otherwise limited, includes known analogs of natural nucleotides that can function in a similar manner to naturally occurring nucleotides. The term “polynucleotide” refers to any form of DNA or RNA, including, for example, genomic DNA; complementary DNA (cDNA), which is a DNA representation of messenger RNA (mRNA), usually obtained by reverse transcription of mRNA or amplification; DNA molecules produced synthetically or by amplification; and mRNA. The term “polynucleotide” encompasses double-stranded nucleic acid molecules, as well as single-stranded molecules. In double-stranded polynucleotides, the polynucleotide strands need not be coextensive (i.e., a double-stranded polynucleotide need not be double-stranded along the entire length of both strands). Polynucleotides are said to be “different” if they differ in structure, e.g., nucleotide sequence.

Polypeptide: The term “polypeptide” refers to an amino acid polymer and is not meant to refer to a specific length of the encoded product and, therefore, encompasses peptides, oligopeptides, and proteins. The polypeptide may also be a naturally occurring allelic or engineered variant of a polypeptide.

Substrate: As used herein, the term “substrate” generally refers to a substrate for an enzyme; i.e., the material on which an enzyme acts to produce a reaction product.

Solid phase: As used herein, a “solid phase” refers to any material that is a solid when employed in the selection methods of the invention.

Synthetic compound: As used herein, the term “synthetic compound” refers to a compound that is not naturally occurring.

Triglyceride: The term “triglyceride” means a chemical moiety comprising a glycerol backbone to which an acyl group is attached at each of the three hydroxyl positions, as depicted below:

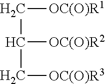

In some instances, the R radical of one or more acyl groups comprises an optionally substituted alkyl group (e.g., a C₄ to C₂₈ optionally substituted alkyl group). A triglyceride may have one or more substituted radicals that link the moiety to another moiety (e.g., a polynucleotide, a selectable marker, and/or a solid phase, as described herein).

Variant: The term “variant” means a lipase comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding one or more amino acids adjacent to and immediately following the amino acid occupying a position.

Wild-type lipase: The term “wild-type” lipase means a lipase expressed by a naturally occurring microorganism, such as a bacterium, yeast, or filamentous fungus found in nature.

Reference to “about” a value or parameter herein includes aspects that are directed to that value or parameter per se. For example, description referring to “about X” includes the aspect “X”. When used in combination with measured values, “about” includes a range that encompasses at least the uncertainty associated with the method of measuring the particular value, and can include a range of plus or minus two standard deviations around the stated value.

As used herein and in the appended claims, the singular forms “a,” “or,” and “the” include plural referents unless the context clearly dictates otherwise. It is understood that the aspects described herein include “consisting” and/or “consisting essentially of” aspects.

Unless defined otherwise or clearly indicated by context, all technical and scientific terms used herein have the same meaning as commonly understood by one of ordinary skill in the art.

### DETAILED DESCRIPTION

Described herein, inter alia, are methods and components used thereof for improving lipase activity. The invention employs in vitro compartmentalization (IVC) for rapid and high throughput enzyme evolution. Instead of relying on a physical link between the genotype and phenotype as implemented in display technologies, IVC links genotype and phenotype by spatial confinement in a single aqueous droplet of a water-in-oil emulsion (See, e.g., Tawfik, D. S. et al., Nat. Biotechnol., 1998, 16(7): 652-656; U.S. Pat. No. 6,489,103; WO 1999/002671; WO 2009/124296).

However, existing IVC screening systems have several disadvantages that make them unsuitable for screening lipases, e.g., requiring a soluble gene-linked substrate that is converted into a product that remains linked to the gene (WO1999/002671), or requiring an insoluble solid-phase cellulosic substrate (WO2009/124296). Additionally, synthetic compounds having a hydrophilic moiety (such a polyanionic nucleic acid) linked to a hydrophobic moiety (such as a triglyceride) typically have technical challenges related to synthesis and use. Nonetheless, the Applicant has surprisingly found that the IVC system as described herein is capable of screening for polypeptides with lipase activity.

Accordingly, described herein is a selection method for enhancing lipase activity. The method makes use of IVC and a collection of synthetic bioconjugate compounds that function as both a selection substrate and a means of encoding a lipase that acts on the substrate. The collection of synthetic compounds includes a collection of polynucleotides that encode for polypeptides (in particular, lipases or lipase derivatives) linked to a collection of triglyceride substrates. Without being bound by theory, the lipase substrate likely anchors the hydrophobic end of the compound to the wall of the oil-encased aqueous droplet from the IVC water-in-oil emulsion, while exposing the hydrophilic end (gene) to components in the aqueous droplet to enable gene expression. An expressed polypeptide having lipase activity can then cleave the lipase substrate from the linked gene, followed by separation of the cleaved and uncleaved synthetic compounds.

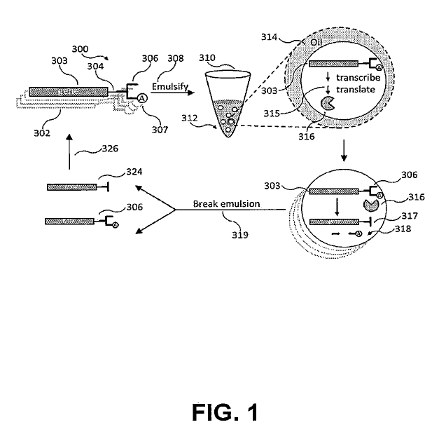

As exemplified by FIG. 1, a negative selection method (300) may employ a collection of polynucleotides (303) encoding for polypeptides, such as a library of synthetic compounds (302) comprising the polynucleotides. The polynucleotides of the library (303) are linked (304) to a triglyceride lipase substrate (306) and are typically mutants that encode variants of an enzyme having lipase activity toward the linked substrate (306). The polynucleotide mutants (303) of the library (302) encoding for the lipase variants may be created using a variety of techniques including mutagenic PCR and DNA library synthesis as set forth in more detail below. PCR amplification using a lipid-modified PCR primer provides one means of linking (304) polynucleotide mutants (303) to a triglyceride substrate (306). Optionally, the triglyceride substrate may be linked to a selectable marker (307) to provide additional means of selectively removing non-hydrolyzed substrate at the end of the process. The polynucleotide library (302) may be emulsified (308) using various oil-surfactants (314) with water to create an emulsion (310) containing aqueous droplets (312) (compartments), each with a compartmentalized synthetic compound. The emulsion is incubated to allow for expression (315) of the polynucleotide mutants (303) into corresponding polypeptides (316).

The expressed polypeptide variants (316) exhibiting lipase activity toward the triglyceride substrate (306) then hydrolyze the substrate (318). Lipase variants with enhanced lipase activity are probabilistically more likely to hydrolyze the DNA-bound triglyceride substrate (306) than lipase variants exhibiting lower activity. A variable incubation temperature and time, as well as use of inhibitors and competitive substrates, enables tuning the assay stringency. After incubation, the emulsion (310) is broken (319). The synthetic compounds with a hydrolyzed triglyceride substrate (324) are then separated from synthetic compounds with a non-hydrolyzed triglyceride substrate (306) using techniques described herein. In the case of a library utilizing a selectable marker (307), separation may be easily accomplished, e.g., using affinity capture. Polynucleotide mutants that encode polypeptide variants with enhanced lipase activity toward the substrate may be subjected to additional rounds (326) of selection to further enhance lipase activity.

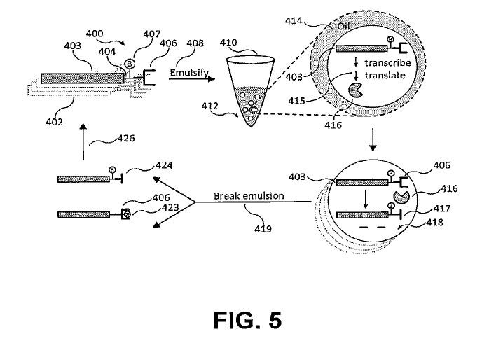

As exemplified by FIG. 5, a positive selection method (400) may employ a collection of polynucleotides (403) encoding for polypeptides, such as a library of synthetic compounds (402) comprising the polynucleotides. The polynucleotides of the library (403) are linked (404) to a triglyceride lipase substrate (406) and are typically mutants that encode variants of an enzyme having lipase activity toward the linked substrate (406). The polynucleotide mutants (403) of the library (402) encoding for the lipase variants may be created using a variety of techniques including mutagenic PCR and DNA library synthesis as set forth in more detail below. PCR amplification using a lipid-modified PCR primer provides one means of linking (404) polynucleotide mutants (403) to a triglyceride substrate (406). The polynucleotides (403) encoding for polypeptides may be linked to a hydrophobic selectable marker (407) to provide additional means of selectively recovering hydrolyzed substrate at the end of the process. The polynucleotide library (402) may be emulsified (408) using various oil-surfactants (414) with water to create an emulsion (410) containing aqueous droplets (412) (compartments), each with a compartmentalized synthetic compound. The emulsion is incubated to allow for expression (415) of the polynucleotide mutants (403) into corresponding polypeptides (416).

The expressed polypeptide variants (416) exhibiting lipase activity toward the triglyceride substrate (406) then hydrolyze the substrate (418). Lipase variants with enhanced lipase activity are probabilistically more likely to hydrolyze the DNA-bound triglyceride substrate (406) than lipase variants exhibiting lower activity. A variable incubation temperature and time, as well as use of inhibitors and competitive substrates, enables tuning the assay stringency. After incubation, the emulsion (410) is broken (419). The synthetic compounds with a hydrolyzed triglyceride substrate (424) are then separated from synthetic compounds with a non-hydrolyzed triglyceride substrate (406) using techniques described herein. Under certain reaction conditions described herein, non-hydrolyzed triglyceride substrate (406) may undergoes hydrophobic collapse (423) thereby sequestering the selectable marker (407) and making the selectable marker unavailable for affinity capture. Polynucleotide mutants that encode polypeptide variants with enhanced lipase activity toward the substrate may be subjected to additional rounds (426) of selection to further enhance lipase activity.

Accordingly, in one aspect is a method of selecting for a polypeptide having lipase activity, the method comprising:
(i) suspending a plurality of synthetic compounds in an aqueous phase, wherein the synthetic compounds individually comprise:
   (a) a polynucleotide encoding for a polypeptide, and
   (b) a lipase substrate (e.g., a triglyceride) linked to said polynucleotide; and
wherein the aqueous phase comprises components for expression of the polypeptide;
(ii) forming a water-in-oil emulsion with the aqueous phase, wherein the synthetic compounds are compartmentalized in aqueous droplets of the emulsion;
(iii) expressing the polypeptides within the aqueous droplets of the emulsion, wherein a polypeptide with lipase activity in an aqueous droplet hydrolyzes one or more synthetic compounds in that droplet; and
(iv) separating the synthetic compounds to recover hydrolyzed and/or non-hydrolyzed synthetic compounds.

### Synthetic Compounds

In one aspect, the synthetic compounds used herein comprise (a) a polynucleotide encoding for a polypeptide, and (b) a lipase substrate (e.g., a triglyceride) linked to said polynucleotide. In some embodiments, a synthetic compound comprises one or more (e.g., two, three) copies of a polynucleotide (having the same or different sequence). In some embodiments, a synthetic compound comprises two polynucleotides (e.g., having the same or different sequence). In some embodiments, a synthetic compound comprises only one copy of one polynucleotide.

### Polynucleotides/Polypeptides

The polynucleotides may comprise a coding sequence for a polypeptide that is, or is derived from, a lipase. Suitable sources of lipases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Available commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

The polynucleotide may comprise a mutated lipase coding sequence that encodes for a lipase variant of a parent lipase. The lipase variants comprise an alteration i.e., a substitution, insertion, and/or deletion, at one or more (e.g., several) positions. Examples of lipase variants are described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

The polynucleotides may comprise suitable control sequences, such as those required for efficient expression of the gene product, for example promoters, enhancers, translational initiation sequences, polyadenylation sequences, splice sites and the like, and as described in detail below.

As described supra, the methods of the present invention may comprise a plurality of synthetic compounds to create a polynucleotide library (e.g., a polynucleotide library encoding a library of lipase variants). In particular embodiments, the libraries have at least about: 10², 10³, 10⁴, 10⁵, 10⁶, 10⁷, 10⁸, 10⁹, 10¹⁰, 10¹¹, 10¹², or 10¹⁴ different synthetic compounds and/or polynucleotides. Generally, the size of the library will be less than about 10¹⁵.

Libraries of polynucleotides can be created in any of a variety of different ways that are well known to those of skill in the art. In particular, pools of naturally occurring polynucleotides can be cloned from genomic DNA or cDNA (Sambrook et al., 1989); for example, phage antibody libraries, made by PCR amplification repertoires of antibody genes from immunized or unimmunized donors have proved very effective sources of functional antibody fragments (Winter et al., 1994; Hoogenboom, 1997). Libraries of genes can also be made by encoding all (see for example Smith, 1985; Parmley and Smith, 1988) or part of genes (see for example Lowman et al., 1991) or pools of genes (see for example Nissim et al., 1994) by a randomized or doped oligonucleotide synthesis.

Libraries can also be made by introducing mutations into a polynucleotide or pool of polynucleotides randomly by a variety of techniques in vivo, including; using mutator strains, of bacteria such as E. coli mutD5 (Liao et al., 1986; Yamagishi et al., 1990; Low et al., 1996); using the antibody hypermutation system of B-lymphocytes (Yelamos et al., 1995). Random mutations can also be introduced both in vivo and in vitro by chemical mutagens, and ionizing or UV irradiation (see Friedberg et al., 1995), or incorporation of mutagenic base analogues (Freese, 1959; Zaccolo et al., 1996). Random mutations can also be introduced into genes in vitro during polymerization for example by using error-prone polymerases (Leung et al., 1989). Further diversification can be introduced by using homologous recombination either in vivo (see Kowalczykowski et al., 1994) or in vitro (Stemmer, 1994a; Stemmer, 1994b)). Libraries of complete or partial genes can also be chemically synthesized from sequence databases or computationally predicted sequences.

Libraries can also be made using DNA recombination like e.g., DNA shuffling. Shuffling between two or more homologous input polynucleotides (starting-point polynucleotides) involves fragmenting the polynucleotides and recombining the fragments, to obtain output polynucleotides (i.e. polynucleotides that have been subjected to a shuffling cycle) wherein a number of nucleotide fragments are exchanged in comparison to the input polynucleotides. DNA recombination or shuffling may be a (partially) random process in which a library of chimeric genes is generated from two or more starting genes. A number of known formats can be used to carry out this shuffling or recombination process. The process may involve random fragmentation of parental DNA followed by reassembly by peR to new full-length genes, e.g. as presented in U.S. Pat. Nos. 5,605,793; 5,811,238; 5,830,721; 6,117,679. In-vitro recombination of genes may be carried out, e.g. as described in U.S. Pat. No. 6,159,687, W098/41623, U.S. Pat. Nos. 6,159,688, 5,965,408, 6,153,510. The recombination process may take place in vivo in a living cell, e.g. as described in WO 97/07205 and WO 98/28416. The parental DNA may be fragmented by DNase I treatment or by restriction endonuclease digests as described by Kikuchi et al (2000a, Gene 236:159-167). Shuffling of two parents may be done by shuffling single stranded parental DNA of the two parents as described in Kikuchi et al (2000b, Gene 243:133-137). A particular method of shuffling is to follow the methods described in Crameri et al, 1998, Nature, 391: 288-291 and Ness et al. Nature Biotechnology 17: 893-896. Another format would be the methods described in U.S. Pat. No. 6,159,687: Examples 1 and 2.

### Lipase Substrates

The lipase substrate of the synthetic compound may be any suitable lipase substrate capable of being hydrolyzed when contacted with a polypeptide having lipase activity. As appreciated by the skilled artisan, selection of an appropriate substrate may depend on the desired lipase activity toward that particular substrate. The lipase substrate may be, for example, a triglyceride, PNP-palmitate, PNP-oleate, 4-methylumbelliferoyl-oleate, propyl laurate, phospholipids (e.g. lecithin), an ester of linoleic acid (e.g., glycerides, phospholipids, galactolipids, waxesters and sterol esters), cutin, a steryl ester, or a wax ester.

In some embodiments, the lipase substrate is a triglyceride. The triglyceride may be linked to a polynucleotide and any suitable position (e.g., 1, 2, and/or 3). In some embodiments, the polynucleotide encoding for a polypeptide is linked at the 2 position of the triglyceride.

Each R moiety on the acyl group of the triglyceride may independently comprise an optionally substituted alkyl group (e.g., a C₄ to C₂₈ optionally substituted alkyl group) such that the resulting triglyceride is a fatty acid linked to the hydroxyl of the glycerol backbone through an ester moiety. The linked fatty acid may be an unsaturated fatty acid (e.g., myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, and docosahexaenoic acid) or a saturated fatty acid (e.g., caprylic acid, capric acid, lauric acid, myristic acid, palmitic acid, stearic acid, arachidic acid, behenic acid, lignoceric acid, cerotic acid). In some embodiments, the triglyceride comprises one optionally substituted R-alkyl group (e.g., R¹; R²; R³). In some embodiments, the triglyceride comprises two optionally substituted R-alkyl groups (e.g., R¹ and R²; R¹ and R³; R² and R³). In some embodiments, the triglyceride comprises three optionally substituted R-alkyl groups (R¹, R², and R³). The optionally substituted R-alkyl groups for these embodiments may be the same or different. In some embodiments, the polynucleotide encoding for a polypeptide is linked to the triglyceride with an R-alkyl group (i.e. the triglyceride comprises an R-alkyl group, wherein the R-alkyl group is substituted with the polynucleotide encoding for a polypeptide).

The lipase substrate may be linked to a polynucleotide using a variety of available conjugation techniques that do not interfere with gene expression, such as linking the triglyceride to the end of each polynucleotide. Standard synthetic techniques may be employed, such as coupling the triglyceride and polynucleotide using a reactive handle (e.g., an activated ester, azide, maleimide, etc.). In one example, a free hydroxyl of the glycerol backbone (e.g., 1,3-dipalmitate) can be modified with a protected mercapto fatty acid linker, deprotected, and finally coupled to a maleimide-linked oligonucleotide primer. The resulting conjugated oligonucleotide-triglyceride is then amplified by PCR with a template polynucleotide sequence to generate the desired synthetic compound. In another example, a 5′-thiol primer is coupled to a triglyceride modified with a maleimide moiety, prior to PCR amplification to afford the desired synthetic compound. Similarly, an amino group on either a modified triglyceride or polynucleotide can be linked to an activated ester (e.g., NHS-ester) of the other partner to produce the desired synthetic compound. Even further still, the conjugation can employ click chemistry, for example, wherein an azide-modified triglyceride is conjugated to an oligonucleotide primer having (i) a terminal alkyne for a copper(I) catalyzed [3+2] azide-alkyne cycloaddition (CuAAC), or (ii) a cyclooctyne derivative, such as dibenzocyclooctyl (DBCO), for a Cu-free click cycloaddition (Jewett et al. Chem. Soc. Rev. 2010 39(4):1272). Accordingly, in some embodiments, the polynucleotide encoding for a polypeptide is linked to the lipase substrate (e.g., via an R-alkyl group of a triglyceride) with a substituted thiol (e.g., thioether), substituted amino (e.g., amido), or triazole moiety.

### Selectable Markers

The synthetic compounds described herein may further comprise a selectable o marker to further distinguish hydrolyzed from non-hydrolyzed synthetic compounds. As used herein, a selectable marker is a chemical moiety that is capable of being cleaved from the synthetic compound by a polypeptide having lipase activity, and which can be detected in a biochemical assay. For example, a selectable marker linked to the lipase substrate (e.g., at the 1, 2, or 3 position of the triglyceride using bioconjugation techniques known in the art and described supra) may be cleaved from the synthetic compound by hydrolysis at the linked acyl chain when contacted with a polypeptide having lipase activity. Non-hydrolyzed synthetic compounds (containing the selectable marker) can then be separated by selective removal from hydrolyzed synthetic compounds. Accordingly, in one embodiment of the methods described herein, the synthetic compound comprises a selectable marker wherein an expressed polypeptide having lipase activity in an aqueous droplet cleaves the selectable marker from one or more of the synthetic compounds in that droplet, thereby allowing selective removal of the non-hydrolyzed synthetic compounds. In another embodiment, the selectable marker is linked to the polynucleotide encoding for a polypeptide. In some embodiments, the selectable marker is sequestered by the non-hydrolyzed synthetic compounds, yet not sequestered by the hydrolyzed synthetic compounds, thereby allowing selective removal of the hydrolyzed synthetic compounds.

The selectable marker may be linked to the synthetic compound using a variety of available conjugation techniques (e.g., those described supra and/or known in the art). The conjugation preferably does not interfere with activity of the lipase on the substrate. In some embodiments, the selectable marker is linked to the lipase substrate or polynucleotide with a substituted thiol (e.g., thioether), substituted amino (e.g., amido), or triazole moiety.

Suitable selectable markers include, but are not limited to affinity tags, where each affinity tag is a member of a binding pair. When used in the methods described herein, a synthetic compound comprising an affinity tag can further aid in separation of hydrolyzed synthetic compound from non-hydrolyzed synthetic compound in step (iv), as non-hydrolyzed compound (containing the affinity tag) can be selectively removed by affinity from hydrolyzed synthetic compound (lacking an affinity tag).

Examples of binding pairs that may be used in the present invention include an antigen and an antibody or fragment thereof capable of binding the antigen (e.g., FLAG tag peptide/Anti-flag tag antibody), the biotin avidin/streptavidin pair (Savage et al., 1994), a calcium-dependent binding polypeptide and ligand thereof (e.g. calmodulin and a calmodulin-binding peptide (Stofko et al., 1992; Montigiani et al.,1996)), pairs of polypeptides which assemble to form a leucine zipper (Tripet et al., 1996), histidines (typically hexahistidine peptides) and chelated Cu²⁺, Zn²⁺ and Ni²⁺, (e.g. Ni-NTA; Hochuli et al., 1987), RNA-binding and DNA-binding proteins (Klug, 1995) including those containing zinc-finger motifs (Klug and Schwabe, 1995) and DNA methyltransferases (Anderson, 1993), and their nucleic acid binding sites. For example, suitable affinity tags include, inter alia, biotin, digoxigenin, dinitrophenyl (DNP), fluorescein, rhodamine (e.g., Texas Red®), and fucose. Biotin and fucose are capable of binding avidin and lectin, respectively, whereas digoxigenin, DNP, fluorescein, and rhodamine are capable of binding to product-specific antibodies. In one embodiment, the synthetic compound comprises a biotin selectable marker. In this embodiment, the one or more hydrolyzed synthetic compounds of step (iv) may be separated from the one or more non-hydrolyzed synthetic compounds with streptavidin (e.g., streptavidin coated microspheres).

### Solid Phases

The synthetic compounds described herein may further comprise a solid phase. Materials useful as solid phases can include: natural polymeric carbohydrates and their synthetically modified, crosslinked, or substituted derivatives, such as agar, agarose, cross-linked alginic acid, chitin, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins, and keratins; natural hydrocarbon polymers, such as latex and rubber; synthetic polymers, such as vinyl polymers, including polyethylene, polypropylene, polystyrene, polyvinylchloride, polyvinyl acetate and its partially hydrolyzed derivatives, polyacrylamides, polymethacrylates, copolymers and terpolymers of the above polycondensates, such as polyesters, polyamides, and other polymers, such as polyurethanes or polyepoxides; porous inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer.

Solid phases generally have a size and shape that permits their suspension in an aqueous medium, followed by formation of a water-in-oil emulsion. Suitable solid phases include microbeads or particles (both termed “microparticles” for ease of discussion). Microparticles useful in the invention can be selected by one skilled in the art from any suitable type of particulate material and include, but are not limited, to those composed of cellulose, Sepharose, polystyrene, polymethylacrylate, polypropylene, latex, polytetrafluoroethylene, polyacrylonitrile, polycarbonate, or similar materials.

In some embodiments, the solid phase is a hydrophobic microbead (e.g., silica beads coated with C4, C8, and C18 alkyl groups, polystyrene, or PS-divinyl benzene). The use of hydrophobic solid phases may further enable separation of the synthetic compounds in step (iv), since compounds that remain attached to the solid phase will more likely be found in the oil phase whereas compounds that have been cleaved from the solid phase by hydrolysis will more likely be found in the aqueous phase.

Preferred microparticles include those averaging between about 0.01 and about 35 microns, more preferably between about 0.5 to 20 microns in diameter, haptenated microparticles, microparticles impregnated by one or preferably at least two fluorescent dyes (particularly those that can be identified after individual isolation in a flow cell and excitation by a laser), ferrofluids (i.e., magnetic particles less than about 0.1 micron in size), magnetic micro spheres (e.g., superparamagnetic particles about 3 microns in size), and other microparticles collectable or removable by sedimentation and/or filtration.

In some embodiments, the solid phase is a nanoparticle, such as a gold nanoparticle. Also contemplated are solid lipid nanoparticles, e.g., as described by Ekambaram et al. (Sci. Revs. Chem. Commun. 2012 2(1), 80-102. The nanoparticles are generally between about 1 to 400 nm in average diameter (e.g., 1 to 100 nm) and include, e.g., spherical colloidal gold, gold nanorods, and urchian shaped nanoparticles.

The solid phases are linked to the synthetic compounds by any means known to those in the art that do not interfere with expression of the linked polynucleotides. For example, an amine modified synthetic compound may be linked to tosyl or carboxylate modified microspheres. Likewise, amino modified microspheres may be coupled to a tosyl or carboxylate modified synthetic compound (or to an amino modified synthetic compound via glutaraldehyde). Hydroxyl, hydrazide or chloromethyl modified microspheres can also be employed, as known in the art. Exemplary synthetic methods for linking the the triglycerides to gold nanoparticles can be found in U.S. Ser. No. 62/143,967, entitled “Methods For Selecting Enzymes Having Enhanced Activity” cofiled with the instant application on Apr. 7, 2015 (See, Example 2).

In some embodiments, the solid phase is linked to the lipase substrate (e.g., to an acyl chain of a triglyceride), thereby anchoring the lipase substrate to the solid phase. In these embodiments, the synthetic compound may be cleaved from the solid phase by hydrolysis of an active lipase. In other embodiments, the solid phase is linked between said lipase substrate and said polynucleotide (e.g., both the lipase substrate and the polynucleotide are linked to the solid phase).

Further examples of linking the solid phase to a polynucleotide (e.g., when linking the solid phase between the lipase substrate and polynucleotide of the synthetic compound) can be found in WO 2009/124296 (the content of which is hereby incorporated by reference).

Also contemplated are methods of making the synthetic compounds described herein, comprising: (i) linking a lipase substrate to a polynucleotide encoding for a polypeptide; and (ii) recovering the synthetic compound. In some embodiments wherein the synthetic compound comprises a selectable marker, the method further comprises linking the lipase substrate to a selectable marker. In some embodiments wherein the synthetic compound comprises a solid phase, the method further comprises linking the lipase substrate to a solid phase.

### Formation of Aqueous Phases Containing Reagents For Polypeptide Expression

Synthetic compounds are combined in an aqueous phase with components for expression of the polypeptide (e.g., in vitro transcription/translation). Such components can be selected for the requirements of a specific system from the following: a suitable buffer, an in vitro transcription/replication system and/or an in vitro translation system containing all the necessary ingredients, enzymes and cofactors, RNA polymerase, nucleotides, transfer RNAs, ribosomes and amino acids (natural or synthetic).

A suitable buffer typically allows the desired components of the biological system to be active and will therefore depend upon the requirements of each specific reaction system. Buffers suitable for biological and/or chemical reactions are known in the art and recipes provided in various laboratory texts, such as Sambrook et al., 1989.

Exemplary in vitro translation systems can include a cell extract, typically from bacteria (Zubay, 1973; Zubay, 1980; Lesley et al., 1991; Lesley, 1995), rabbit reticulocytes (Pelham and Jackson, 1976), or wheat germ (Anderson et al., 1983). Many suitable systems are commercially available (for example from Promega) including some which will allow coupled transcription/translation (all the bacterial systems and the reticulocyte and wheat germ TNT™ extract systems from Promega). The mixture of amino acids used may include synthetic amino acids if desired, to increase the possible number or variety of proteins produced in the library. This can be accomplished by charging tRNAs with artificial amino acids and using these tRNAs for the in vitro translation of the proteins to be selected (Ellman et al., 1991; Benner, 1994; Mendel et al., 1995).

### Formation of Emulsions

Emulsions may be produced from any suitable combination of immiscible liquids to enable a suitable platform for compartmentalizing the synthetic compounds described herein. In some embodiments, the emulsion is suitable for expressing the polypeptides (e.g., within an aqueous droplet), and those expressed polypeptides having lipase activity are capable of hydrolyzing one or more synthetic compounds in that droplet.

Preferably the emulsion of the present invention has water (containing the biochemical components described supra) as the phase present in the form of finely divided droplets (the disperse, internal or discontinuous phase) and a hydrophobic, immiscible liquid (an oil) as the matrix in which these droplets are suspended (the nondisperse, continuous or external phase). Such emulsions are termed water-in-oil (W/O).

The emulsion may be stabilized by addition of one or more surface-active agents (surfactants). These surfactants are termed emulsifying agents and act at the water/oil interface to prevent (or at least delay) separation of the phases. Many oils and many emulsifiers can be used for the generation of water-in-oil emulsions; a recent compilation listed over 16,000 surfactants, many of which are used as emulsifying agents (Ash and Ash, 1993). Suitable oils include light white mineral oil and non-ionic surfactants (Schick, 1966) such as sorbitan monooleate (Span™80; ICI) and polyoxyethylenesorbitan monooleate (Tween™ 80; ICI).

The use of anionic surfactants may also be beneficial. Suitable surfactants include sodium cholate and sodium taurocholate. Particularly preferred is sodium deoxycholate, preferably at a concentration of 0.5% w/v, or below. Inclusion of such surfactants can in some cases increase the expression of the polynucleotides and/or the activity of the enzymes/enzyme variants. Addition of some anionic surfactants to a non-emulsified reaction mixture completely abolishes translation. During emulsification, however, the surfactant may be transferred from the aqueous phase into the interface and activity is restored. Addition of an anionic surfactant to the mixtures to be emulsified ensures that reactions proceed only after compartmentalization.

Creation of an emulsion generally requires the application of mechanical energy to force the phases together. There are a variety of ways of doing this that utilize a variety of mechanical devices, including stirrers (such as magnetic stir-bars, propeller and turbine stirrers, paddle devices and whisks), homogenizers (including rotor-stator homogenizers, high-pressure valve homogenizers and jet homogenizers), colloid mills, ultrasound and ‘membrane emulsification’ devices (Becher, 1957; Dickinson, 1994). Accordingly, in one aspect is a method of preparing an emulsion described herein, comprising (i) suspending the plurality of synthetic compounds in the aqueous phase, and (ii) mixing the suspension of (i) with an oil.

Aqueous droplets formed in water-in-oil emulsions are generally stable with little if any exchange of polynucleotides or enzymes/enzyme variants between droplets. The technology exists to create emulsions with volumes all the way up to industrial scales of thousands of liters (Becher, 1957; Sherman, 1968; Lissant, 1974; Lissant, 1984).

The preferred droplet size will vary depending upon the precise requirements of any individual selection process that is to be performed according to the present invention. In all cases, there will be an optimal balance between polynucleotide library size, the required enrichment and the required concentration of components in the individual droplets to achieve efficient expression and reactivity of the enzymes/enzyme variants.

The processes of expression preferably occur within each individual droplet provided by the present invention. Both in vitro transcription and coupled transcription/translation become less efficient at sub-nanomolar DNA concentrations. Because of the requirement for only a limited number of DNA molecules to be present in each droplet, this therefore sets a practical upper limit on the possible droplet size. In some embodiments, the average volume of the droplets is between about 1 altoliter and about 1 nanoliter, inclusive (e.g., between about 10 altoliter and about 50 femtoliter, or about 0.5 femtoliter and about 10 femtoliter). The average diameter of the aqueous droplets typically falls within about 0.05 μm and about 100 μm, inclusive. In some embodiment, aqueous droplets in the emulsion have an average diameter between about 0.1 μm and about 50 μm, about 0.2 μm and about 25 μm, about 0.5 μm and about 10 μm, about 1 μm and about 5 μm, about 2 μm and about 4 μm, or about 3 μm and about 4 μm, inclusive. In certain embodiments, the mean volume of the droplets is less than 5.2×10⁻¹⁶ m³ (corresponding to a spherical droplet of diameter less than 10 μm), less than 6.5×10⁻¹⁷ m³ (corresponding to a spherical droplet of diameter less than 5 μm), less than or about 4.2×10⁻¹⁸ m³ (2 μm), or less than or about 9×10⁻¹⁸ m³ (2.6 μm).

The effective polynucleotide concentration in the droplets may be artificially increased by various methods that will be well-known to those versed in the art. These include, for example, the addition of volume excluding chemicals such as polyethylene glycols (PEG) and a variety of gene amplification techniques, including transcription using RNA polymerases including those from bacteria such as E. coli (Roberts, 1969; Blattner and Dahlberg, 1972; Roberts et al., 1975; Rosenberg et al., 1975), eukaryotes e. g. (Weil et al., 1979; Manley et al., 1983) and bacteriophage such as T7, T3 and SP6 (Melton et al., 1984); the polymerase chain reaction (peR) (Saiki et al., 1988); Q-beta replicase amplification (Miele et al., 1983; Cahill et al., 1991; Chetverin and Spirin, 1995; Katanaev et al., 1995); the ligase chain reaction (LCR) (Landegren et al., 1988; Barany, 1991); self-sustained sequence replication system (Fahy et al., 1991) and strand displacement amplification (Walker et al., 1992). Even gene amplification techniques requiring thermal cycling such as PCR and LCR could be used if the emulsions and the in vitro transcription or coupled transcription/translation systems are thermostable (for example, the coupled transcription/translation systems could be made from a thermostable organism such as Thermus aquaticus).

Increasing the effective local nucleic acid concentration enables larger droplets to be used effectively. This allows a preferred practical upper limit for most applications to the droplet volume of about 2.2×10⁻¹⁴ m³ (corresponding to a sphere of diameter 35 μm).

The droplet size should be sufficiently large to accommodate all of the required components of the biochemical reactions that are needed to occur within the droplet, in addition to the synthetic compound. In vitro, both transcription reactions and coupled transcription/translation reactions typically employ a total nucleotide concentration of about 2 mM. For example, in order to transcribe a gene to a single short RNA molecule of 500 bases in length, this would require a minimum of 500 molecules of nucleotides per droplet (8.33×10⁻²² moles). In order to constitute a 2 mM solution, this number of molecules must be contained within a droplet of volume 4.17×10⁻¹⁹ liters (4.17×10⁻²² m³ which if spherical would have a diameter of 93 nm.

Furthermore, the ribosomes necessary for the translation to occur are themselves approximately 20 nm in diameter. Hence, the in some embodiments lower limit for droplets is a diameter of approximately 0.1 μm (100 nm).

The size of emulsion droplets may be varied simply by tailoring the emulsion conditions used to form the emulsion according to requirements of the selection system. The larger the droplet size, the larger is the volume that will be required to emulsify a given polynucleotide library, since the ultimately limiting factor will be the size of the droplet and thus the number of droplets possible per unit volume. In some embodiments, the emulsion comprises at least about 10², 10³, 10⁴, 10⁵, 10⁶, 10⁷, 10⁸, 10⁹, 10¹⁰, 10¹¹, 10¹², or 10¹⁵ droplets/mL of emulsion.

Depending on the complexity and size of the library to be screened, it may be beneficial to form an emulsion such that in general 1 or less than 1 synthetic compound is included in each droplet of the emulsion. The number of synthetic compounds per droplet is governed by the Poisson distribution. Accordingly, if conditions are adjusted so that there are, on average, 0.1 synthetic compound per droplet, then, in practice, approximately: 90% of droplets will contain no synthetic compound, 9% of droplets will contain 1 synthetic compound, and 1% of droplets will contain 2 or more synthetic compounds. In practice, average values of about 0.1 to about 0.5, more preferably about 0.3, synthetic compounds per droplet provide emulsions that contain a sufficiently high percentage of droplets having 1 synthetic compound per droplet, with a sufficiently low percentage of droplets having 2 or more synthetic compounds per droplet. This approach will generally provide the greatest power of resolution. Where the library is larger and/or more complex, however, this may be less practical; it may be preferable to include several synthetic compound together and rely on repeated application of the method of the invention to achieve sorting of the desired activity. In some embodiments, no more than 70%, 60%, 50%, 40%, 30%, 20%, 15%, 10% or 5% of the aqueous droplets of the water-in-oil emulsion comprise more than one synthetic compound

Theoretical studies indicate that the larger the number of polynucleotide mutants created the more likely it is that a corresponding encoded polypeptide will be created with the properties desired (See, e.g., Perelson and Oster, 1979 for a description of how this applies to repertoires of antibodies). Recently it has also been confirmed practically that larger phage-antibody repertoires do indeed give rise to more antibodies with better binding affinities than smaller repertoires (Griffiths et al., 1994). To ensure that rare variants are generated and thus are capable of being selected, a large library size is generally desirable.

Using the present system, at an aqueous droplet diameter of 2.6 μm, a repertoire size of at least 10¹¹ can readily be sorted using 1 ml aqueous phase in a 20 ml emulsion.

### Expression, Separation and Further Processing

The emulsion is maintained for a sufficient time under conditions suitable for expression of the polypeptides. The active lipases act to hydrolyze the lipase substrate attached to the polynucleotides, and selectable marker, if present. When using a triglyceride substrate, lipase activity thus results in the cleavage of one or more acyl chains in the substrate attached to the polynucleotides encoding active lipase/lipase variants via hydrolysis. By attenuating the expression conditions using the teachings described herein, the gene coding sequences for those lipases with enhanced enzymatic activity can be distinguished from those having less activity.

In some embodiments, expression occurs by incubating the emulsion at about 25° C. to about 60° C. (e.g., about 25° C. to about 50° C., about 30° C. to about 40° C.) for about 1 hour to about 24 hours (e.g., about 1 hour to about 12 hours, about 1 hour to about 5 hours, or about 1 hour to about 2 hours).

In some embodiments, the aqueous phase is separated from the oil phase (e.g., prior to step (iv)) by any suitable technique, such as, for example chemically-induced coalescence and/or centrifugation.

The hydrolyzed synthetic compounds may be separated from the non-hydrolyzed synthetic compounds using any of a number of conventional techniques. For example, separation of non-hydrolyzed from hydrolyzed substrate may be accomplished, e.g., by using C18 magnetic beads (e.g. Dynabeads® RPC 18, Thermo Fisher Scientific, Inc.). Magnetic silica beads coated with C4, C8, and C18 alkyl groups are routinely used to separate hydrophobic species (e.g., non-hydrolyzed synthetic compounds having fatty acid chains intact). Separation my also occur by removing non-hydrolyzed compounds through binding to silica or anion exchange, or charge switch media, as known in the art. Further, as described supra, separation can further be aided when the synthetic compound comprises a selectable marker, where, e.g., antibodies, lectin, or streptavidin can bind to the marker and remove non-hydrolyzed compounds by affinity capture.

In some embodiments, the recovered hydrolyzed and/or non-hydrolyzed synthetic compounds results in the compounds being substantially pure. With respect to hydrolyzed synthetic compounds, “substantially pure” intends a recovered preparation of hydrolyzed synthetic compounds that contains no more than 15% impurity, wherein impurity intends non-hydrolyzed synthetic compounds. With respect to non-hydrolyzed synthetic compounds, “substantially pure” intends a recovered preparation of non-hydrolyzed synthetic compounds that contains no more than 15% impurity, wherein impurity intends hydrolyzed synthetic compounds. In some variations, substantially pure hydrolyzed synthetic compounds or non-hydrolyzed synthetic compounds may contain no more than 10% impurity, or no more than 5% impurity, or no more than 3% impurity, or no more than 1% impurity, or no more than 0.5% impurity.

The collection of separated synthetic compounds (hydrolyzed and/or non-hydrolyzed) may be further analyzed. For example, after each round of selection, the enrichment of the pool of polynucleotides for those encoding a lipase of interest can be analyzed, e.g., by non-compartmentalized sequencing reactions known in the art. In one embodiment, the method further comprises analyzing the polynucleotide sequence (e.g., via sequencing) of one or more of the separated synthetic compounds of step (iv), such as one or more of the hydrolyzed synthetic compounds, and/or one or more of the non-hydrolyzed compounds.

The selected pool can be amplified and/or cloned into a suitable expression vector for propagation and/or expression, as described below, using techniques known in the art. In one embodiment, the method further comprises amplifying one or more polynucleotides of the one or more hydrolyzed synthetic compounds of step (iv). In another embodiment, the method further comprises amplifying one or more polynucleotides of the one or more non-hydrolyzed synthetic compounds of step (iv).

The polynucleotides of the separated synthetic compounds may also be subjected to subsequent, possibly more stringent rounds of sorting in iteratively repeated steps, reapplying the method of the invention either in its entirety or in selected steps only. By tailoring the conditions appropriately, synthetic compounds encoding lipases having a better optimised activity may be generated after each round of selection. Accordingly, in some embodiments, the method is reiterated wherein the polynucleotides of the separated synthetic compounds (e.g., the amplified polynucleotides from the hydrolyzed synthetic compounds) are used in a new plurality of synthetic compounds as described in step (i), and steps (i)-(iv) are repeated with said new plurality of synthetic compounds. If desired, further genetic variation can be introduced into the polynucleotides prior to repeating the method, using, e.g. error-prone polymerase chain reaction (PCR) and/or other techniques described supra. Accordingly, in one embodiment, the method further comprising introducing an alteration to (e.g., via mutagenizing) one or more polynucleotides of the separated synthetic compounds of step (iv).

### Nucleic Acid Constructs and Expression Vectors

In some embodiments, the methods described herein further comprise cloning one or more polynucleotides of the separated synthetic compounds from step (iv) into a nucleic acid construct or expression vector. RNA and/or recombinant protein can be produced from the individual clones for further purification and assay (as described below). Recombinant selected using the methods of the invention can be employed for any application for which the native enzyme is employed. Thus, in some embodiments, the methods further comprise expressing one or more of polynucleotides from the separated synthetic compounds of step (iv) (e.g., expressing a polynucleotide of a hydrolyzed synthetic compound to produce a polypeptide with lipase activity).

The nucleic acid constructs comprise a polynucleotide encoding a polypeptide or variant described herein operably linked to one or more control sequences that direct the expression of the coding sequence in a suitable host cell under conditions compatible with the control sequences.

The polynucleotide may be manipulated in a variety of ways to provide for expression of a polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide which is recognized by a host cell for expression of the polynucleotide. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xyIA and xyIB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol 3-hydroxypropionate dehydrogenase/glyceraldehyde-3-phosphate 3-hydroxypropionate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator sequence is operably linked to the 3′-terminus of the polynucleotide encoding the variant. Any terminator that is functional in the host cell may be used.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate 3-hydroxypropionate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader sequence is operably linked to the 5′-terminus of the polynucleotide encoding the variant. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol 3-hydroxypropionate dehydrogenase/glyceraldehyde-3-phosphate 3-hydroxypropionate dehydrogenase (ADH2/GAP).

The control sequence may also be a polyadenylation sequence, a sequence operably linked to the 3′-terminus of the variant-encoding sequence and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Thermomyces lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of the variant and the signal peptide sequence is positioned next to the N-terminus of the propeptide sequence.

It may also be desirable to add regulatory sequences that regulate expression of the variant relative to the growth of the host cell. Examples of regulatory systems are those that cause expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the variant would be operably linked with the regulatory sequence.

Recombinant expression vectors comprise a polynucleotide encoding a polypeptide or variant described herein, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene.

The vector preferably contains an element(s) that permits integration of the vector into the host cell's genome or autonomous replication of the vector in the cell independent of the genome.

For integration into the host cell genome, the vector may rely on the polynucleotide's sequence encoding the variant or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMß1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

In some embodiments, the methods described herein further comprise transforming one or more polynucleotides of the separated synthetic compounds from step (iv) (e.g., a nucleic acid construct or expression vector comprising the polynucleotide) into a recombinant host cell. A construct or vector comprising a polynucleotide is introduced into a host cell so that the construct or vector is maintained as a chromosomal integrant or as a self-replicating extra-chromosomal vector. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carisbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Thermomyces lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

In some embodiments, the methods described herein further comprise cultivating a recombinant host cell described supra under conditions suitable for expression of the polypeptide, and optionally recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a whole fermentation broth comprising the polypeptide is recovered.

The polypeptide may be purified by a variety of procedures known in the art including, but not limited to, chromatography (e.g., ion exchange, affinity, hydrophobic, chromatofocusing and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

The present invention may be further described by the following numbered paragraphs:
[1] A method of selecting for a polypeptide having lipase activity, the method comprising:
   (i) suspending a plurality of synthetic compounds in an aqueous phase, wherein the synthetic compounds individually comprise:
      (a) a polynucleotide encoding for a polypeptide, and
      (b) a lipase substrate linked to said polynucleotide; and
   wherein the aqueous phase comprises components for expression of the polypeptide;
   (ii) forming a water-in-oil emulsion with the aqueous phase, wherein the synthetic compounds are compartmentalized in aqueous droplets of the emulsion;
   (iii) expressing the polypeptides within the aqueous droplets of the emulsion, wherein a polypeptide with lipase activity in an aqueous droplet hydrolyzes one or more synthetic compounds in that droplet; and
   (iv) separating the synthetic compounds to recover hydrolyzed and/or non-hydrolyzed synthetic compounds.
[2] The method of paragraph 1, wherein the lipase substrate is a triglyceride.
[3] The method of paragraph 1 or 2, wherein the plurality of synthetic compounds comprises at least about 10⁶ different synthetic compounds (e.g., at least about 10¹⁰, 10¹², or 10¹⁴ different synthetic compounds).
[4] The method of any one of the preceding paragraphs, wherein no more than 20% of the aqueous droplets of the water-in-oil emulsion comprise more than one synthetic com pound.
[5] The method of any one of the preceding paragraphs, wherein each synthetic compound comprises only one copy of one polynucleotide.
[6] The method of any one of the preceding paragraphs, wherein the emulsion comprises at least about 10⁶ aqueous droplets/mL of emulsion (e.g., at least about 10⁹, 10¹², or 10¹⁵ aqueous droplets/mL of emulsion).
[7] The method of any one of the preceding paragraphs, wherein the aqueous droplets in the emulsion have an average diameter between about 0.05 μm and about 100 μm, inclusive (e.g., between about 0.1 μm and about 50 μm, about 0.2 μm and about 25 μm, about 0.5 μm and about 10 μm, or about 1 μm and about 5 μm, inclusive).
[8] The method of any one of the preceding paragraphs, wherein the aqueous droplets in the emulsion have an average volume of between about 1 altoliter and about 1 nanoliter, inclusive (e.g., between about 10 altoliter and about 50 femtoliter, or about 0.5 femtoliter and about 10 femtoliter).
[9] The method of any one of the preceding paragraphs, wherein the polynucleotide encoding for a polypeptide is linked at the 2 position of the triglyceride.
[10] The method of any one of the preceding paragraphs, wherein the polynucleotide encoding for a polypeptide is linked to the lipase substrate with a substituted thiol (e.g., thioether), substituted amino (e.g., amido), or triazole moiety.
[11] The method of any one of the preceding paragraphs, wherein the synthetic compounds further comprise a selectable marker.
[12] The method of paragraph 11, wherein the selectable marker is linked to the lipase substrate (e.g., at the 1 or 3 position of the triglyceride).
[13] The method of paragraph 12, wherein the selectable marker is linked to the lipase substrate with a substituted thiol (e.g., thioether), substituted amino (e.g., amido), or triazole moiety.
[14] The method of any one of paragraphs 11-13, wherein an expressed polypeptide having lipase activity in an aqueous droplet cleaves the selectable marker from one or more of the synthetic compounds in that droplet, thereby allowing selective removal of the non-hydrolyzed synthetic compounds of step (iv).
[15] The method of paragraph 11, wherein the selectable marker is linked to the polynucleotide encoding for a polypeptide.
[16] The method of paragraph 15, wherein the selectable marker is linked to polynucleotide with a substituted thiol (e.g., thioether), substituted amino (e.g., amido), or triazole moiety.
[17] The method of any one of paragraphs 11, 15, or 16, wherein the selectable marker is o sequestered by the non-hydrolyzed synthetic compounds, thereby allowing selective removal of the hydrolyzed synthetic compounds of step (iv).
[18] The method of any one paragraphs 11-17, wherein the selectable marker is an affinity tag.
[19] The method of paragraph 18, wherein the affinity tag comprises biotin.
[20] The method of paragraph 19, wherein the hydrolyzed synthetic compounds of step (iv) are separated from the non-hydrolyzed synthetic compounds with streptavidin (e.g., streptavidin coated microspheres).
[21] The method of any one of the preceding paragraphs, wherein the synthetic compounds individually comprise a solid phase.
[22] The method of paragraph 21, wherein the solid phase is linked to said lipase substrate.
[23] The method of paragraph 22, wherein the solid phase is linked between said lipase substrate and said polynucleotide.
[24] The method of any one of paragraphs 21-23, wherein the solid phase is a microbead or particle.
[25] The method of paragraph 24, wherein the solid phase is a hydrophobic microbead.
[26] The method of any one of paragraphs 21-23, wherein the solid phase is a gold nanoparticle.
[27] The method of any one of the preceding paragraphs, comprising separating the aqueous phase from the oil phase (e.g., via chemically-induced coalescence and/or centrifugation) prior to step (iv).
[28] The method of any one of the preceding paragraphs, wherein the recovered hydrolyzed and/or non-hydrolyzed synthetic compounds are substantially pure.
[29] The method of any one of the preceding paragraphs, further comprising analyzing the polynucleotide sequence (e.g., via sequencing) of one or more of the separated synthetic compounds of step (iv).
[30] The method of any one of the preceding paragraphs, further comprising amplifying one or more polynucleotides of the hydrolyzed synthetic compounds of step (iv).
[31] The method of any one of the preceding paragraphs, further comprising amplifying one or more polynucleotides of the non-hydrolyzed synthetic compounds of step (iv).
[32] The method of paragraph 30 or 31, wherein the amplified one or more polynucleotides are used in a new plurality of synthetic compounds as described in step (i), and steps (i)-(iv) are repeated with said new plurality of synthetic compounds.
[33] The method of any one of the preceding paragraphs, further comprising introducing an alteration to (e.g., mutagenizing) one or more polynucleotides of the separated synthetic compounds of step (iv).
[34] The method of paragraph 33, wherein the one or more altered polynucleotides are used in a new plurality of synthetic compounds as described in step (i), and steps (i)-(iv) are repeated with said new plurality of synthetic compounds.
[35] The method of any one of the preceding paragraphs, further comprising expressing one or more of polynucleotides from the separated synthetic compounds of step (iv) (e.g., expressing a polynucleotide of a hydrolyzed synthetic compound to produce a polypeptide with lipase activity).
[36] The method of any one of the preceding paragraphs, further comprising cloning one or more polynucleotides of the separated synthetic compounds from step (iv) into an expression vector.
[37] The method of paragraph 36, further comprising transforming said expression vector into a recombinant host cell.
[38] The method of paragraph 37, further comprising cultivating the recombinant host cell under conditions suitable for expression of the polypeptide, and optionally recovering the polypeptide.
[39] A synthetic compound comprising:
   (a) a polynucleotide encoding for a polypeptide; and
   (b) a lipase substrate linked to said polynucleotide.
[40] The synthetic compound of paragraph 39, wherein the lipase substrate is a triglyceride.
[41] The synthetic compound of paragraph 39 or 40, which comprises only one copy of one polynucleotide.
[42] The synthetic compound of paragraph 40 or 41, wherein the polynucleotide encoding for a polypeptide is linked at the 2 position of the triglyceride.
[43] The synthetic compound of any one of paragraphs 39-42, wherein the polynucleotide encoding for a polypeptide is linked to the lipase substrate with a substituted thiol (e.g., thioether), substituted amino (e.g., amido), or triazole moiety.
[44] The synthetic compound of any one of paragraphs 39-43, wherein the polypeptide has lipase activity.
[45] The synthetic compound of any one of paragraphs 39-44, wherein the polypeptide is a lipase variant.
[46] The synthetic compound of any one of paragraphs 39-45, further comprising a selectable marker.
[47] The synthetic compound of paragraph 46, wherein the selectable marker is linked to the lipase substrate (e.g., at the 1 or 3 position of the triglyceride).
[48] The synthetic compound of paragraph 46 or 47, wherein the selectable marker is linked to the lipase substrate with a substituted thiol (e.g., thioether), substituted amino (e.g., amido), or triazole moiety.
[49] The synthetic compound of any one of paragraphs 46-48, wherein the selectable o marker is capable of being cleaved from the compound when contacted with a polypeptide having lipase activity.
[50] The synthetic compound of paragraph 46, wherein the selectable marker is linked to the polynucleotide encoding for a polypeptide.
[51] The synthetic compound of paragraph 50, wherein the selectable marker is linked to polynucleotide with a substituted thiol (e.g., thioether), substituted amino (e.g., amido), or triazole moiety.
[52] The synthetic compound of any one paragraphs 46-51, wherein the selectable marker is an affinity tag.
[53] The synthetic compound of paragraph 52, wherein the affinity tag comprises biotin.
[54] The synthetic compound of any one of paragraphs 39-53, further comprising a solid phase.
[55] The synthetic compound of paragraph 54, wherein the solid phase is linked to said lipase substrate.
[56] The synthetic compound of paragraph 55, wherein the solid phase is linked between said lipase substrate and said polynucleotide.
[57] The synthetic compound of any one of paragraphs 54-56, wherein the solid phase is a microbead or particle.
[58] The synthetic compound of paragraph 57, wherein the solid phase is a hydrophobic microbead.
[59] The synthetic compound of any one of paragraphs 54-56, wherein the solid phase is a gold nanoparticle.
[60] The synthetic compound of any one of paragraphs 39-59, which is capable of being hydrolyzed when contacted with a polypeptide having lipase activity.
[61] A method of making the synthetic compound of any one of paragraphs 39-60, comprising:
   (i) linking a lipase substrate to a polynucleotide encoding for a polypeptide; and
   (ii) recovering the synthetic compound.
[62] The method of paragraph 61, further comprising: linking the lipase substrate to a selectable marker.
[63] The method of paragraph 61 or 62, further comprising linking the lipase substrate to a solid phase.
[64] A polynucleotide library comprising a plurality of different synthetic compounds according to any one of paragraphs 39-60.
[65] The polynucleotide library of paragraph 64, wherein the plurality of synthetic compounds comprises at least about 10⁶ different synthetic compounds (e.g., at least about 10¹⁰, 10¹², or 10¹⁴ different synthetic compounds).
[66] A water-in-oil emulsion comprising the polynucleotide library of paragraph 64 or 65, wherein the synthetic compounds are compartmentalized in aqueous droplets of the emulsion.
[67] The emulsion of paragraph 66, wherein no more than 20% of the aqueous droplets of the water-in-oil emulsion comprises more than one synthetic compound.
[68] The emulsion of paragraph 66 or 67, further comprising components for expression of the polypeptide in the aqueous droplets.
[69] The emulsion of any one of paragraphs 66-68, further comprising an emulsifying agent.
[70] The emulsion of any one of paragraphs 66-69, comprising at least about 10⁶ aqueous droplets/mL of emulsion (e.g., at least about 10⁹, 10¹², or 10¹⁵ aqueous droplets/mL of emulsion).
[71] The emulsion of any one of paragraphs 66-70, wherein the aqueous droplets have o an average diameter between about 0.05 μm and about 100 μm, inclusive (e.g., between about 0.1 μm and about 50 μm, about 0.2 μm and about 25 μm, about 0.5 μm and about 10 μm, or about 1 μm and about 5 μm, inclusive).
[72] The emulsion of any one of paragraphs 66-71, wherein the aqueous droplets have an average volume of between about 1 altoliter and about 1 nanoliter, inclusive (e.g., between about 10 altoliter and about 50 femtoliter, or about 0.5 femtoliter and about 10 femtoliter).
[73] The emulsion of any one of paragraphs 66-72, wherein the emulsion is suitable for expressing the polypeptides within the aqueous droplets.
[74] The emulsion of any of one of paragraphs 66-73, wherein the expressed polypeptides having lipase activity are capable of hydrolyzing one or more synthetic compounds in that droplet.
[75] A method of making the emulsion of any one of paragraphs 66-74, comprising:
   (i) suspending the plurality of synthetic compounds in the aqueous phase; and
   (ii) mixing the suspension of (i) with an oil.

The following examples are provided by way of illustration and are not intended to be limiting of the invention.

### EXAMPLES

Chemicals used as buffers and substrates were commercial products of at least reagent grade.

### Example 1

### Synthesis of LSS1 (1)

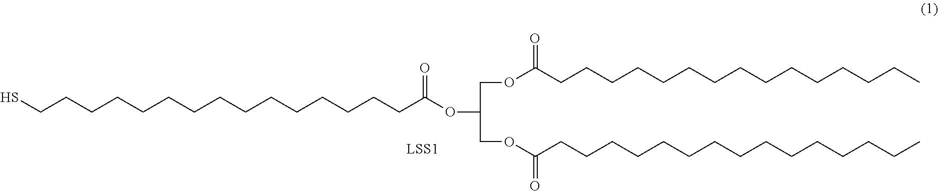 [SMILES: CCCCCCCCCCCCCCCC(=O)OCC(COC(=O)CCCCCCCCCCCCCCC)OC(=O)CCCCCCCCCCCCCCCS]

LSS1 features a 16-mercaptohexadecanoic acid (FA-SH) in the 2 position of the triglyceride (1). To limit byproduct formation, it was decided to protect the thiol of the FA-SH before coupling it to the diglyceride. The 4-methoxytrityl (Mmt) was chosen over the standard trityl protection group since it is significantly more acid labile, which could be important in deprotection of the triglyceride product. The conditions chosen for introducing the protecting group was inspired by a procedure found in Mourtas et al. Tetrahedron Lett. 2001, 42, 6965-6967, whereas conditions chosen for the subsequent coupling to form the triglyceride (1) were similar to a procedure found in Whitten et al. Tetrahedron 2012, 68, 5422-5428.

### FA-SMmt

16-Mercaptohexadecanoic acid (366 mg, 1.27 mmol) and 4-methoxytrityl chloride (392 mg, 1 eq) was dissolved in DCM-DMF (1:1, 6 mL). Diisopropylethylamine, DIPEA (0.5 mL, 2.3 eq) was added to the mixture. Stirred at RT for 2 h. TLC (1:1 EtOAc-heptane) then showed full conversion. Rf (Mmt-Cl)=0.70; Rf (product)=0.56. The reaction mixture was evaporated in vacuum, re-dissolved in CHCl₃ and purified by flash chromatography (eluting initially with EtOAc-hexane 1:4, later 1:1). Product containing fractions were identified by TLC, pooled and evaporated to dryness. Yield: 458 mg (64%) of a white wax.

¹H NMR (400 MHz, CDCl₃, selected signals in ppm): 6.79 (d, 2H, Mmt CH next to —OMe), 3.77 (s, 3H, —OMe), 2.34 (t, 2H, —SCH₂—), 2.14 (t, 2H, —CH₂COOH), 1.64-1.60 (m, 2H, —SCH₂CH_{2—).}

¹³C NMR (100 MHz, CDCl₃, selected signals in ppm): 180.4 (—COOH), 113.1 (Mmt CH next to —OMe), 65.9 (Mmt quaternary C), 55.2 (—OMe), 34.1 (—SCH₂—), 32.1 (—CH₂COOH), 24.7 (—SCH₂CH₂—).

### TG-SMmt

FA-SMmt (458 mg, 0.817 mmol) and glyceryl 1,3-dipalmitate (511 mg, 1.1 eq) were dissolved in DCM-THF (1:1, 10 mL) and cooled to 0° C. 1-Ethyl-3-(3-dimethylaminopropyl)carbodiimide hydrochloride, EDAC (391 mg, 2.5 eq) and DMAP (10 mg, 0.1 eq) were added. The mixture was stirred for 4 h while slowly warming up to RT, after which TLC (1:5 EtOAc-hexane) showed almost full conversion. Rf (product)=0.60. The reaction mixture was evaporated, re-dissolved in CHCl₃ (5 mL) and purified by flash chromatography, eluting with EtOAc-heptane (1:9). Yield: 779 mg (86%) of a white waxy oil.

¹H NMR (400 MHz, CDCl₃, selected signals in ppm): 6.83 (d, 2H, Mmt CH next to —OMe), 5.39-5.28 (m, 1H, H-2), 4.34 (dd, 2H, H-⅓), 4.18 (dd, 2H, H-⅓), 3.80 (s, 3H, —OMe), 2.37-2.32 (m, 6H, —CH₂COO—), 2.18 (t, 2H, —CH₂S—), 1.66-1.61 (m, 6H, —CH₂CH₂COO—), 0.91 (t, 6H, —CH₃).

¹³C NMR (100 MHz, CDCl₃, selected signals in ppm): 113.0 (Mmt CH next to —OMe), 68.9 (C-2), 62.1 (C-⅓), 55.2 (—OMe), 34.1 (—CH₂S—), 32.0 (—CH₂COO—), 24.9 (—CH₂CH₂CO—), 22.7 (—CH₂CH₃), 14.2 (—CH₃).

### LSS1 (1)

TG-SMmt (90 mg, 0.081 mmol) was dissolved in DCM (1.5 mL). TFA (18 uL) and triethylsilane (TES, 15 uL, 1.1 eq) was added. Stirring under N₂ at room temperature. The mixture turned yellowish-brown upon addition of TFA and again colorless after addition of the TES scavenger. After 30 min, TLC (EtOAc-heptane 1:19) showed full conversion. Rf (SM)=0.44, Rf (product)=0.56, Rf (trityl)=0.60. After staining with H₂SO₄, the SM and trityl appeared as yellow spots and the product as a brown spot (non-UV active). The reaction mixture was then evaporated in vacuum and purified by flash chromatography using an eluent going from hexane to EtOAc-hexane (1:10). Fractions containing pure product were pooled and evaporated to yield 70 mg (91%) of a white wax.

¹H NMR (400 MHz, CDCl₃, selected signals in ppm): 5.29 (m, 1H, H-2), 4.32 (dd, 2H, H-⅓), 4.17 (dd, 2H, H-⅓), 2.55 (dt, 2H, —CH₂SH), 2.33 (t, 6H, —CH₂CO—), 1.67-1.59 (m, 8H, —CH₂CH₂CO— and —CH₂CH₂SH), 0.90 (t, 6H, —CH₃).

¹³C NMR (100 MHz, CDCl₃, selected signals in ppm): 68.9 (C-2), 62.1 (C-⅓), 31.9 (—CH₂CH₂CH₃), 22.7 (—CH₂CH₃), 14.1 (—CH₃).

### Example 2

### Synthesis of LSS1b (2)

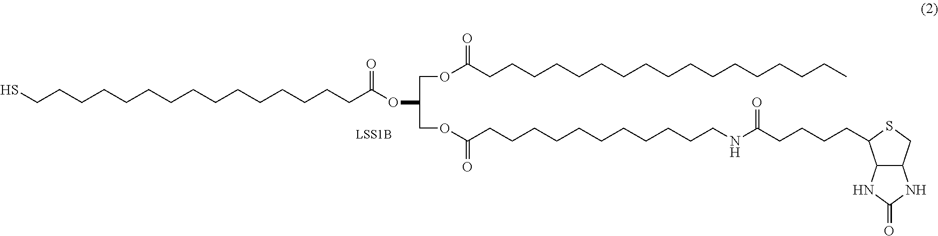

For the synthesis of LSS1b, 12-aminododecanoic acid (FA-NH2) was Mmt-protected similar to FA-SH (supra). Following a procedure for trityl protection of amino acids, in which the carboxylic acid is temporarily protected as a TMS-ester, the desired FA-NHMmt was produced in good yield (Barlos et at. J. Org Chem. 1982, 47, 1324-1326). The coupling reactions now proceeded using the same protocol as for synthesis of LSS1. Since model studies indicated that biotin-NHS reacts fast and chemoselectively with amino nucleophiles, the DG-NHMmt was coupled with FA-SMmt, before both protecting groups were removed simultaneously. Finally, coupling to biotin-NHS yielded the desired LSS1b (2).

### Biotin-NHS

Biotin (325 mg, 1.33 mmol) was dissolved in DMF (10 mL) with gentle heating. After cooling to RT, N,N-diisopropylcarbodiimide (DIPCDI, 250 uL, 1 eq), pyridine (105 uL, 1 eq) and N-hydroxysuccinimide (HNS, 200 mg, 1.3 eq) were added. The mixture was stirred at RT ON, then filtered and concentrated in vacuum. The resulting solid was re-crystallized in iPrOH (50 mL), by first dissolving it using gentle heating, then placing the solution to precipitate at 4° C. The precipitated product was filtered, washed with cold iPrOH and dried in vacuum to yield 280 mg (62%) of white crystals.

¹H NMR (400 MHz, DMSO-d6, selected signals in ppm): 6.42 (s, 1H, NH), 6.36 (s, 1H, NH), 4.33-4.29 (m, 1H, —CHNH—), 4.18-4.13 (m, 1H, —CHNH—), 3.15-3.08 (m, 1H, —CH—S—), 2.82 (s, 4H, —CH₂— NHS), 2.68 (t, 2H, —CH₂COO—).

¹³C NMR (100 MHz, DMSO-d6, selected signals in ppm): 170.7 (CO NHS), 169.4 (CO ester), 163.2 (CO carbamide), 61.5 (—CHNH—), 59.6 (—CHNH—), 55.7 (—CH—S—), 30.5 (—CH₂COO—), 25.9 (—CH₂— NHS).

### FA-NHMmt

12-Aminododecanoic acid (1.08 g, 5 mmol) was suspended in CHCl₃-MeCN (5:1, 18 mL) and chloro trimethylsilane (TMS-Cl, 0.63 mL, 1 eq) was added. The mixture was heated to reflux (65° C.) under N₂ for 2 h which remained as a suspension. After cooling to RT, triethylamine (1.39 mL, 2 eq) was added. Then addition of Mmt-Cl (1.54 g, 1 eq) dissolved in CHCl₃ (10 mL). The turbid orange solution was stirred at RT ON. MeOH (25 mmol, 1.0 mL) was then added. The orange solution slowly turned yellow. TLC (EtOAc-heptane, 1:3) confirmed full conversion of Mmt-Cl (Rf 0.40) to product (Rf 0.15). Evaporated to an oil, of which 1.7 g is purified by flash chromatography. It is dissolved in CHCl₃ (10 mL) and eluted through a 120 g silica flash cartridge using EtOAc-heptane (1:3, 600 mL and then 2:3, 750 mL). The product containing fractions were identified by TLC, pooled and evaporated to yield 1.4 g, 57% of yellow viscous oil.

¹H NMR (400 MHz, CDCl₃, selected signals in ppm): 6.83 (d, 2H, Mmt CH next to —OMe), 3.81 (s, 3H, —OMe), 2.36 (t, 2H, —CH₂COOH), 2.16 (t, 2H, —NHCH₂—), 1.68-1.61 (m, 2H, —CH₂CH₂COOH), 1.53-1.46 (m, 2H, —NHCH₂CH₂—).

¹³C NMR (100 MHz, CDCl₃, selected signals in ppm): 55.2 (—OMe), 43.7 (—NHCH₂—), 34.1 (—CH₂COOH).

### DG-NHMmt

FA-NHMmt (585 mg, 1.2 mmol) was dissolved in THF-DCM (1:1, 12 mL) and the solution cooled to 0° C. on ice. 1-Stearoyl-rac-glycerol (430 mg, 1 eq), EDAC (570 mg, 2.5 eq) and DMAP (26 mg, 0.2 eq) were added. The mixture was stirred under N₂ at 0° C. for 2 h, and then at RT for 2 h. Some precipitation. TLC (EtOAc-hexane 1:3) then showed full conversion. Rf (product)=0.47. The reaction mixture was evaporated and re-dissolved in CHCl₃ (2 mL). Purification by flash chromatography, eluting with a hexane-EtOAc gradient going from pure hexane to 20% EtOAc. The product is eluted with 20% EtOAc. The product containing fractions are evaporated to yield 405 mg, 41%.

¹H NMR (400 MHz, CDCl₃, selected signals in ppm): 6.83 (d, 2H, Mmt CH next to —OMe), 4.23-4.09 (m, 5H, glycerin backbone), 3.80 (s, 3H, —OMe), 2.37 (t, 4H, —CH₂CO—), 2.13 (t, 2H, —CH₂NH—), 1.67-1.61 (m, 4H, —CH₂CH₂COOH), 1.55-1.45 (m, 2H, —NHCH₂CH_{2—),} 0.91 (t, 3H, —CH₃).

¹³C NMR (100 MHz, CDCl₃, selected signals in ppm): 173.9 (—CO—), 113.0 (Mmt CH next to —OMe), 70.3 (Mmt quaternary C), 68.4 (C-2), 65.06 (C-⅓), 55.2 (—OMe), 43.6 (—CH₂NH—), 34.1 (—CH₂CO—), 24.9 (—CH₂CH₂CO—), 22.7 (—CH₂CH₃), 14.1 (—CH₃).

### TG-(SMmt)-NHMmt

DG-NHMmt (200 mg, 0.24 mmol), EDAC (119 mg, 2.5 eq), DMAP (6 mg, 0.2 eq) and FA-SMmt (136 mg, 1 eq) were dissolved in DCM-THF (3:2, 5 mL). The solution was stirred under N₂ at 0° C. for 1 h and then allowed to warm to RT. TLC (EtOAc-heptane 1:3) after 18 h showed almost full conversion with Rf (product)=0.5. The reaction mixture was evaporated in vacuum and purified by flash chromatography, eluting with EtOAc-heptane 1:7. This yielded 279 mg, 87% pure target product.

¹H NMR (400 MHz, CDCl₃, selected signals in ppm): 6.84-6.82 (m, 4H, Mmt CH next to —OMe), 5.31-5.26 (m, 1H, H-2), 4.31 (dd, 2H, H-⅓), 4.17 (dd, 2H, H-⅓), 3.81 (s, 3H, —OMe), 3.80 (s, 3H, —OMe), 2.33 (t, 6H, —CH₂CO—), 2.18-2.11 (m, 4H, —CH₂NH—+—CH₂S—), 0.91 (t, 3H, —CH₃).

¹³C NMR (100 MHz, CDCl₃, selected signals in ppm): 173.3 (—COO—), 172.9 (—COO—), 113.1 (Mmt CH next to —OMe), 113.0 (Mmt CH next to —OMe), 70.3 (Mmt quaternary C), 68.9 (C-2), 65.9 (Mmt quaternary C), 62.1 (C-⅓), 55.2 (—OMe), 55.1 (—OMe), 43.6 (—CH₂NH—), 34.2 (—CH₂CO—), 34.1 (—CH₂CO—), 24.9 (—CH₂CH₂CO—), 24.8 (—CH₂CH₂CO—), 22.7 (—CH₂CH₃), 14.1 (—CH₃).

### LSS1b (2)

TG-(SMmt)-NHMmt (279 mg, 0.208 mmol) was dissolved in DCM (5 mL). TFA (0.1 mL) and TES (56 uL, 1.6 eq) were added. The clear solution was stirred at 0° C. under N₂. The reaction turns yellow and then colorless. TLC (MeOH-EtOAc 1:9) soon showed full conversion with Rf (product) 0.25 (brown after staining with H₂SO₄) and Rf (Mmt)=0.85 (yellow after staining with H₂SO₄). The mixture was then concentrated in vacuum to yield 355 mg deprotected TG(SH)NH₂ which was used directly for biotin coupling. Hence, the residue was re-suspended (not fully soluble) in DMF (2 mL) and triethylamine (142 uL, 5 eq) and biotin-NHS (84 mg, 1.2 eq) were added. The mixture was stirred at 40° C. for 1 h after which TLC (MeOH-DCM, 1:9) showed full conversion. Rf (SM) 0.27 and Rf (LSS1b) 0.38. After evaporation in vacuum, the residue was fully dissolved in DCM (4 mL) and purified by flash chromatography, eluting first with DCM, then with MeOH-DCM (1:13). The product containing fractions were identified by TLC, pooled and evaporated to yield 56 mg (26% over two steps) LSS1b as a white solid.

¹H NMR (400 MHz, CDCl₃, selected signals in ppm): 5.60 (t, 1H, —NHCO—), 5.31-5.26 (m, 1H, H-2), 4.57-4.52 (m, 1H, —CHNH— biotin), 4.40-4.35 (m, 1H, —CHNH— biotin), 4.35-4.27 (m, 2H, H-⅓), 4.17 (dd, 2H, H-⅓), 3.25 (q, 2H, —CH₂NH—), 3.19 (q, 1H, —CH—S— biotin), 2.95 (dd, 1H, —CH₂—S— biotin), 2.76 (d, 1H, —CH₂—S— biotin), 2.54 (q, 2H, —CH₂SH), 2.37-2.29 (m, 6H, —CH₂OOO—), 2.25-2.18 (m, 2H, —NHCOCH₂—), 0.90 (t, 3H, —CH₃).

¹³C NMR (100 MHz, CDCl₃, selected signals in ppm): 173.34, 173.32, 172.9, 172.7 (4*-CO—), 163.2 (—NHCONH—), 68.9 (C-2), 62.1 (C-⅓), 61.8 (—NHCH— biotin), 60.1 (—NHCH— biotin), 55.2 (—CH—S— biotin), 40.5 (—CH₂—S— biotin), 39.6 (—CH₂NHCO—), 36.0 (—NHCOCH_{2—),} 34.2 (—CH₂CH₂SH), 34.1 (3*-CH₂COO—), 31.9 (—CH₂CH₂CH₃), 22.7 (—CH₂CH₃), 14.1 (—CH₃).

LC-MS, m/z (C₅₉H₁₀₉N₃O₈S₂, monoisotopic mass 1051.77 Da): 1052.71 [M+H]⁺, 1074.87 [M+Na]⁺.

### Example 3

### Synthesis of LSS1.2b (3)

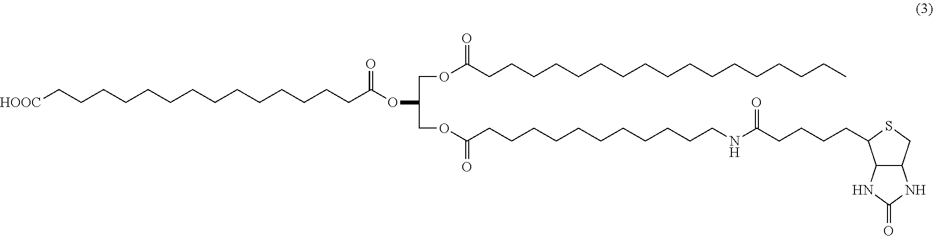

The carboxylic acid terminated biotinylated triglyceride LSS1.2b was synthesized in a similar manner to LSS1 b, using mono-tBu protected hexadecandioic acid as building block. Synthesis of this followed a procedure by Høeg-Jensen et al. (WO2011000823). The Mmt group is considerably more acid labile than tBu and was removed with our standard conditions (2% TFA in DCM), whereas tBu required neat formic acid. Removing both protecting groups in the same step (with formic acid) of course saves one step, but the subsequent coupling to biotin-NHS was troublesome due to low solubility of the —COOH, —NH₂ terminated triglyceride. An often seen byproduct after coupling to biotin-NHS is free NHS (singlet at 2.75 ppm), which seems to co-elute with LSS1.2b. This is however easily removed by a simple aqueous extraction.

### Hexadecandioic Acid Mono-tBu Ester

Hexadecandioc acid (4.28 g, 14.9 mmol) was suspended in toluene (50 mL) and the mixture heated to reflux. N,N-dimethylformamide di-tBu-acetal (10 mL, 42 mmol) was added dropwise under N₂ and reflux continued ON. The mixture was then evaporated to dryness, re-suspended in DCM-EtOAc (1:1, 50 mL) and stirred for 15 min at RT. After removing solids by filtration, the solution was evaporated to dryness again, re-suspended in DCM (5 mL), and cooled on ice for 10 min, before more solid was removed by filtration. The solution was finally evaporated to dryness to yield 1.49 g of crude product, which was further purified by re-crystallization from heptane (20 mL) to yield 802 mg pure product (16%).

¹H NMR (_{400 MHz}, CDCl₃, selected signals in ppm): 2.37 (t, 2H, —CH₂COOH), 2.22 (t, 2H, —CH₂COOtBu), 1.69-1.62 (m, 2H, —CH₂CH₂COOH), 1.62-1.55 (m, 2H, —CH₂CH₂COOtBu).

### TG-(COOtBu)-NHMmt

DG-NHMmt (484 mg, 0.58 mmol) was dissolved in DCM-THF (3:2, 12 mL) and the solution cooled to 0° C. Hexadecanoic acid mono-tBu ester (202 mg, 1 eq) was added, followed by EDAC (270 mg, 2.4 eq) and DMAP (15 mg). The mixture was stirred under N₂ at 0° C. for 1 h, then at RT ON. TLC (EtOAc-heptane 1:4) showed several products. The mixture was then evaporated and purified by flash chromatography, eluting with EtOAc-heptane (1:7). The target compound was identified in fractions 1-8, which were pooled and evaporated, yielding 423 mg pure product (63%).

¹H NMR (400 MHz, CDCl₃, selected signals in ppm): 6.83 (d, 2H, Mmt CH next to —OMe), 5.31-5.26 (m, 1H, H-2), 4.31 (dd, 2H, H-⅓), 4.17 (dd, 2H, H-⅓), 3.80 (s, 3H, —OMe), 2.37-2.30 (m, 6H, —CH₂COO—), 2.22 (t, 2H, —CH₂COOtBu), 2.13 (t, 2H, —CH₂NH—), 1.46 (s, 9H, tBu), 0.90 (t, 3H, —CH₃).

¹³C NMR (100 MHz, CDCl₃, selected signals in ppm): 113.0 (Mmt CH next to —OMe), 68.9 (C-2), 62.1 (C-⅓), 55.2 (—OMe), 43.6 (—CH₂NH—), 35.6 (—CH₂COOtBu), 28.1 (tBu), 14.1 (—CH₃).

### TG-(COOtBu)-NH₂

TG-(COOtBu)-NHMmt (423 mg, 0.37 mmol) was dissolved in TFA-DCM (2% TFA v/v, 14 mL), to which TES (95 uL, 1.6 eq) was added at 0° C. The color changed from yellow to colorless after 20 min. TLC then showed full conversion. The mixture was evaporated and purified by flash chromatography, eluting with a gradient of MeOH in DCM (3% to 10% v/v). Product-containing fractions were pooled and concentrated to yield 290 mg (90%).

¹H NMR (400 MHz, CDCl₃, selected signals in ppm): 5.31-5.26 (m, 1H, H-2), 4.31 (dd, 2H, H-⅓), 4.17 (dd, 2H, H-⅓), 2.36-2.29 (m, 6H, —CH₂COO—), 2.22 (t, 2H, —CH₂COOtBu), 1.46 (s, 9H, tBu), 0.90 (t, 3H, —CH₃).

### TG-(COOtBu)-NHBiotin

TG-(COOtBu)-NH₂ (140 mg, 0.14 mmol assuming TFA salt) was dissolved in DMF (2 mL) and added biotin-NHS (66 mg, 1.4 eq) and triethylamine (85 uL, 4.3 eq). The mixture was stirred 1 h at 40° C. TLC (MeOH-DCM, 1:9) then showed full conversion with Rf (SM) 0.40, Rf (biotin-NHS) 0.48 and Rf (product) 0.20 after staining with KMnO₄. The mixture was concentrated and purified by flash chromatography, eluting with 4% MeOH in DCM. Yield: 110 mg (71%).

¹H NMR (400 MHz, CDCl₃, selected signals in ppm): 5.76 (t, 1H, —NHCO—), 5.32-5.24 (m, 1H, H-2), 4.57-4.52 (m, 1H, —CHNH— biotin), 4.37-4.27 (m, 3H, —CHNH— biotin and H-⅓), 4.16 (dd, 2H, H-⅓), 3.25 (q, 2H, —CH₂NH—), 3.19 (q, 1H, —CH—S— biotin), 2.95 (dd, 1H, —CH₂—S— biotin), 2.76 (d, 1H, —CH₂—S— biotin), 2.37-2.29 (m, 6H, 3*—CH₂COO— glyceride), 2.22 (t, 4H, —CH₂COOtBu and —CH₂CONH—), 1.46 (s, 9H, tBu), 0.90 (t, 3H, —CH₃).

### LSS1.2b (3)

TG-(COOtBu)-NHBiotin (50 mg, 0.045 mmol) was added formic acid (1 mL) and TES (20 uL, 2.9 eq). It was not fully soluble, but formed a suspension. It was difficult to follow the reaction progress by TLC due to the formic acid. However, evaporation and NMR analysis after 1 h showed both product and SM. Hence the reaction was continued (new HCOOH and TES was added) for 5 h at RT. NMR then showed almost full conversion. The mixture was evaporated and purified by flash chromatography, eluting with a gradient of MeOH in DCM (from 7% to 20% v/v). Yield: 19 mg pure LSS1.2b (40%).

¹H NMR (400 MHz, CDCl₃, selected signals in ppm): 5.89 (t, 1H, —NHCO—), 5.34-5.26 (m, 1H, H-2), 4.56-4.52 (m, 1H, —CHNH— biotin), 4.38-4.34 (m, 1H, —CHNH— biotin), 4.30 (dd, 2H, H-⅓), 4.19-4.13 (m, 2H, H-⅓), 3.24 (q, 2H, —CH₂NH—), 3.19 (q, 1H, —CH—S— biotin), 2.96 (dd, 1H, —CH₂—S— biotin), 2.75 (d, 1H, —CH₂—S— biotin), 2.37-2.29 (m, 8H, 3*—CH₂COO— glyceride and —CH₂COOH), 2.26-2.20 (m, 2H, —NHCOCH₂—), 0.90 (t, 3H, —CH₃).

¹³C NMR (100 MHz, CDCl₃, selected signals in ppm): 68.9 (C-2), 62.3 (C-⅓), 62.1 (C-⅓), 61.9 (—CHNH— biotin), 60.2 (—CHNH— biotin), 55.3 (—CH—S— biotin), 40.6 (—CH₂—S— biotin), 39.6 (—CH₂NHCO—), 35.9 (—NHCOCH₂—), 34.4, 34.3, 34.1, 34.0 (4*—CH₂COO—), 31.9 (—CH₂CH₂CH₃), 22.7 (—CH₂CH₃), 14.1 (—CH₃).

### Example 4

### Synthesis of LSS1pb (4)

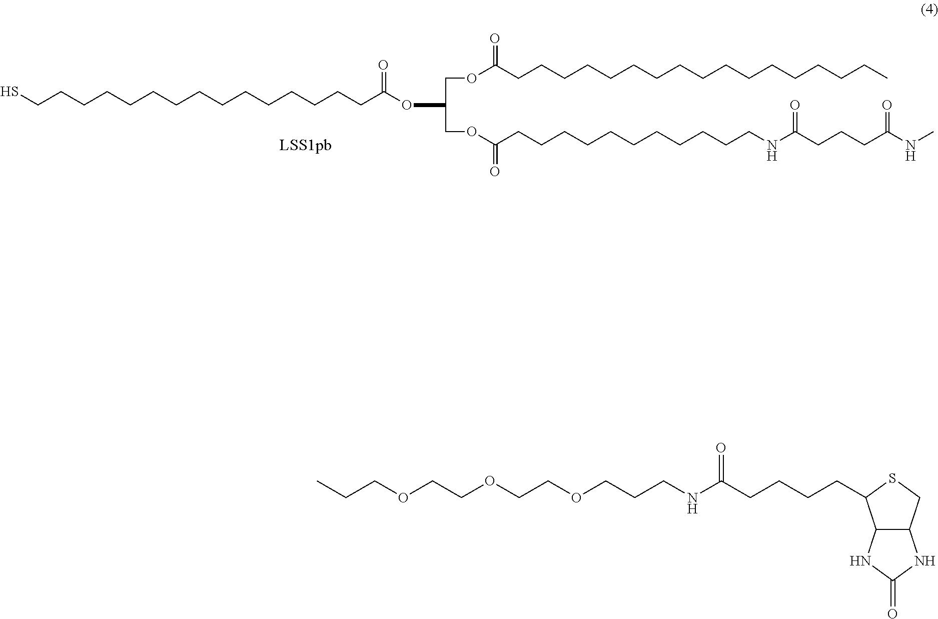

LSS1pb (4, pegylated biotin) was synthesized similar to LSS1b supra, with the exception that a pegylated biotin (Novabiochem 8.51029.0001) was used as starting material. This was converted to the active NHS-ester, which finally was coupled to the deprotected TG(SH)NH₂.

pBiotin-NHS

N-Biotinyl-NH-(PEG)₂-COOH (318 mg, 0.45 mmol) was dissolved in DMF (1 mL) and DIPCDI (85 uL, 1 eq), pyridine (35 uL, 1 eq) and HNS (66 mg, 1.3 eq) were added. The mixture was stirred at RT ON. TLC (DCM-MeOH, 9:1) indicated good conversion with Rf (NHS) 0.40 and Rf (product) 0.24. The crude product was evaporated in vacuum, re-dissolved and purified by flash chromatography, eluting with DCM-MeOH (9:1) through a 25 g silica flash cartridge. Product-containing fractions were identified by TLC (DCM-MeOH, 9:1), pooled and evaporated in vacuum, yielding 175 mg (59%) of the target product.

¹H NMR (400 MHz, CDCl₃, selected signals in ppm): 6.73 (t, 1H, —NHCO—), 6.68 (t, 1H, —NHCO—), 6.40 (s, 1H, NH biotin), 5.71 (s, 1H, NH biotin), 4.54-4.46 (m, 1H, —CHNH— biotin), 4.35-4.26 (m, 1H, —CHNH— biotin), 3.68-3.50 (m, 12H, —CH₂O—), 3.36-3.28 (m, 4H, —CH₂NH—), 3.14 (q, 1H, —CH—S— biotin), 2.85 (s, 4H, —CH₂— NHS), 2.68 (t, 2H, —CH₂COO—).

¹³C NMR (100 MHz, CDCl₃, selected signals in ppm): 61.8 (—CHNH— biotin), 60.2 (—CHNH— biotin), 55.6 (—CH—S— biotin), 40.5 (—CH₂—S— biotin), 36.1, 34.5 (—CH₂—CONH—), 30.2 (—CH₂COO—), 25.8 (—CH₂— NHS).

### LSS1pb (4)

TG(SH)NH2 (54 mg, 0.057 mmol assuming TFA salt) was dissolved in DMF (1 mL). pBiotin-NHS (61 mg, 1.6 eq) was likewise dissolved in DMF (1 mL). Two solutions were combined and triethylamine (30 uL, 3.8 eq) was added. The mixture was stirred for 2 h at 40° C. after which TLC (DCM-MeOH, 9:1) showed full conversion with Rf(TG(SH)NH2) 0.38 and Rf(LSS1pb) 0.29. Purification by flash chromatography, eluting with 6-10% MeOH in DCM. Yield: 41 mg (53%) of LSS1pb as a white solid.

¹H NMR (400 MHz, CDCl₃, selected signals in ppm): 5.31-5.25 (m, 1H, H-2), 4.57-4.51 (m, 1H, —CHNH— biotin), 4.38-4.27 (m, 3H, —CHNH— biotin and H-⅓), 4.16 (dd, 2H, H-⅓), 3.70-3.55 (m, 12H, —CH₂O—), 3.40-3.31 (m, 4H, —OCH₂CH₂CH₂NH—), 2.54 (dd, 2H, —CH₂SH), 2.37-2.30 (m, 6H, —CH₂COO—), 0.90 (t, 3H, —CH₃).

¹³C NMR (100 MHz, CDCl₃, selected signals in ppm): 68.9 (H-2), 61.8 (—CHNH— biotin), 60.3 (—CHNH— biotin), 55.5 (—CH—S— biotin), 40.4 (—CH₂—S— biotin), 34.1 (—CH₂CH₂SH), 24.5 (—CH₂SH), 14.2 (—CH₃).

### Example 5

### Linking Substrate to DNA

The triglyceride compound 2 (Example 2) was conjugated to an oligonucleotide primer as follows: Amino-modified oligonucleotide 5′-/5AmMC6/AAAAA ACGGA GCGAA CCACT TATC (SEQ ID NO: 7)-3′ was synthesized at 10 μmole scale by Integrated DNA Technologies, Inc. (Coralville, Iowa USA) and purified by standard desalting. The oligonucleotide primer then was precipitated with hexadecyltrimethylammonium chloride (CTAC) and resuspended in THF.

Compound 2 was modified with the heterobifunctional linker containing a maleimide moiety and N-hydroxysuccinimide flanking a PEG spacer (NHS-PEGn-maleimide, where n=2, 6, 12, or 24; Thermo Fisher Scientific Inc., Rockford, Ill. USA) in THF with Hunig's base. To this was added the resuspended oligonucleotide primer above. The resulting conjugated product was purified using ethanol precipitation, spin column filtration, size exclusion chromatography, and HPLC.

The resulting conjugated product above was used in two separate PCR with either template A (SEQ ID NO: 1, containing the coding sequence of SEQ ID NO: 2, and encodes for the polypeptide of SEQ ID NO: 3) or template B (SEQ ID NO: 4, containing the coding sequence of SEQ ID NO: 5, and encodes for the polypeptide of SEQ ID NO: 6) as follows: A 50 μL aq. reaction was assembled containing 0.5 μM Primer 1 (5′-LSS1b-(SEQ ID NO: 7)-3′), 0.5 μM Primer 2 (5′-GCAGC TAGGG CTGTT GTCTT TA (SEQ ID NO: 8)-3′), 500 pg of template A or template B, and 25 μL Q5® High-Fidelity 2× Master Mix (New England Biolabs, Ipswich, Mass. USA). The reaction was activated at 98° C. for 30 s and then thermal cycled 28 times (98° C. for 5 s, 65° C. for 20 s, 72° C. for 20 s) followed by a final extension at 72° C. The resulting 903 bp biotinylated triglyceride amplicon was purified from residual PCR components using the Agencourt AMPure XP system (Beckman Coulter, Inc., Indianapolis, Ind. USA) according to the manufacture's protocol.

### Example 6

### Emulsion Formation and Polypeptide Expression

The triglyceride amplicon of Example 5 was emulsified using the following procedure: A 125 μL aq. in vitro transcription/translation (IVTT) reaction was assembled on ice using the PURExpress® In Vitro Protein Synthesis Kit (New England Biolabs, Ipswich, Mass. USA). The IVTT reaction contained 50 μL PURExpress® Tube A, 2.5 μL PURExpress® Disulfide Bond Enhancer, 2.5 μL Murine RNase Inhibitor (#M0314), 36.25 μL PURExpress® Tube B, and ˜10⁸ molecules of the triglyceride amplicons, of which approximately 2.5% were derived from SEQ ID NO: 1 (encoding for the wild-type Thermomyces lanuginosus lipase of SEQ ID NO: 3) and 97.5% derived from SEQ ID NO: 4 (encoding for the catalytically inactive Thermomyces lanuginosus lipase SEQ ID NO: 6). All components except the triglyceride amplicon were purchased from New England Biolabs, Ipswich, Mass. USA. The cold 125 μL IVTT reaction was combined with 375 μL of room temperature 3M Novec HFE-7500, 1% Pico-Surf 1 (The Dolomite Centre Ltd., Royston, UK) in a 2 mL microfuge tube (Eppendorf AG, Hamburg Germany) with a 7 mm Stainless Steel Bead (Qiagen, Venlo, Limburg). The tube was agitated in a TissueLyser (Qiagen, Venlo Limburg) at 15 Hz for 10 s followed by 30 Hz for 30 s. Emulsions were incubated at 37° C. for 1 to 24 hr to allow polypeptide expression and triglyceride hydrolysis. For some tests, the emulsion temperature was raised to 50° C. for an additional hour to investigate the effects of higher temperature on triglyceride hydrolysis.

After expression/hydrolysis, the aqueous fraction was recovered as follows: Methyl Arachidonyl Fluorophosphonate (MAFP, Abcam plc., Cambridge UK) was added to a final concentration of 75 μM to inhibit any further lipase activity. 750 μL of Pico-Break 1 (The Dolomite Centre Ltd., Royston, UK) was added to the emulsion and then inverted 10 times. Another 750 μL of Pico-Break 1 was added followed by vortexing for 30 s. The tube was centrifuged 2,000×g at 4° C. for 1 min. The top aqueous fraction was carefully removed by pipetting and transferred to a clean tube.

Synthetic compound in the recovered aqueous fraction was purified from protein, surfactant, ribonucleic acid, salt and other contaminants using the Universal Quick-DNA™ Miniprep Kit (Zymo Research Corp., Irvine, Calif. USA) according to the manufacturer's protocol. Briefly, 100 μL of the recovered aqueous fraction was combined with 100 μL of BioFluid & Cell Buffer and 10 μL of Proteinase K (20 mg/mL). The reaction was mixed by vortexing 10 s and then incubated at 55° C. for 10 min. 630 μL of Genomic Binding Buffer was then added and vortexed 10 s. The mixture transferred to a Zymo-Spin™ IIC-XL Column and centrifuged at 14,000×g for 1 min. The column was washed sequentially with 400 μL DNA Pre-Wash Buffer, 700 μL g-DNA Wash Buffer, and finally 200 μL of g-DNA Wash Buffer. For each wash step, the column was centrifuged at 14,000×g for 1 min., discarding the flow through after each centrifugation. The purified synthetic compounds were eluted in 50 μL of DNA Elution Buffer.

### Example 7

### Separation of Hydrolyzed and Non-Hydrolyzed Synthetic Compounds Using Affinity Capture

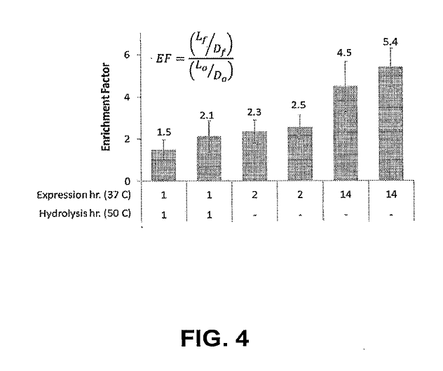

Synthetic compounds with polynucleotides encoding enzymes with low or no activity toward the substrate retain the affinity tag and were selectively removed by affinity capture as follows: Dynabeads® MyOne™ Streptavidin C1 magnetic beads (Thermo Fisher Scientific, Inc.) were washed once in a 100-μL volume of IDTE (10 mM Tris, pH 8.0, 0.1 mM EDTA) using a Kingfisher automated magnetic particle processor (Thermo Scientific, Inc.). The washed beads were resuspended 50 μL of elution from Example 3 containing purified synthetic compounds were combined and 50 μL of 2× binding buffer (20 mM Tris, pH 8.0, 0.2 mM EDTA, 2 M NaCl, 20% PEG-8000). The synthetic compounds were collected on the beads for 1-24 hr at 20-60° C. All synthetic compounds adsorb onto the beads, but only non-hydrolyzed synthetic compound that retain the affinity tag specifically and nearly-irreversibly bind to the streptavidin beads. The beads are then moved to a fresh volume of 100 μL of IDTE, termed “elution” where hydrolyzed synthetic compounds resulting from active lipase that do not retain the affinity tag are eluted from the beads. Non-hydrolyzed synthetic compounds encoding inactive or less-active lipase variants retain the affinity tag and remain bound to the beads. The beads are then moved to a second fresh volume of 100 μL of IDTE and released. Measurement of enrichment is performed with a technique such as quantitative PCR (qPCR) or droplet digital PCR (ddPCR) which can accurately quantitate the relative abundance of active and inactive alleles. Enrichment in FIG. 4 is presented as the Enrichment Factor, which is the quotient of the final ratio of active to inactive alleles (L_{final} and D_{final}, respectively) and the starting ratio of active to inactive alleles (L₀ and D₀, respectively).

### Example 8

### Separation of Hydrolyzed and Non-Hydrolyzed Synthetic Compounds Without Using Affinity Capture (Prophetic)

For synthetic compounds without a selectable marker, separation of non-hydrolyzed from hydrolyzed substrate may be accomplished, e.g., by using C18 magnetic beads (e.g. Dynabeads® RPC 18, Thermo Fisher Scientific, Inc.). Magnetic silica beads coated with C4, C8, and C18 alkyl groups are routinely used to separate and recover hydrophobic species.

The triglyceride compound 1 (Example 1) is conjugated to an oligonucleotide primer and PCR amplified as described in Example 5. The amplicon is then emulsified and expressed as described in Example 6. The eluted aqueous phase is then treated with Magnetic silica beads to remove non-hydrolyzed synthetic compounds encoding inactive or less active lipase.

### Example 9

### Separation of Hydrolyzed and Non-Hydrolyzed Synthetic Compounds Using Affinity Capture (Prophetic)

Affinity capture of hydrolyzed substrates may be accomplished by modification of the polynucleotide or linker with biotin or other selectable marker instead of modifying the triglyceride with biotin or other selectable marker. At certain surfactant concentrations, biotin is sequestered by hydrophobic interactions with the non-hydrolyzed triglyceride substrate linked to the polynucleotide and is not effectively captured. For example, 30 min binding at room temperature to streptavidin beads in 1×B&W (5 mM Tris, pH 7.5, 0.5 mM EDTA, 1M NaCl, 0.01% Tween 20) yields <2% capture of non-hydrolyzed substrate while providing ≥98% capture 5′ biotinylated DNA.

An amino and biotin-modified oligonucleotide primer is conjugated to the triglyceride compound 1 (Example 1) and PCR amplified as described in Example 5. The amplicon is then emulsified and expressed as described in Example 6. The eluted aqueous phase is then treated with magnetic streptavidin beads in the presence of surfactants to capture hydrolyzed synthetic compounds encoding active lipase.

### Example 10

### Linking Substrate to DNA For Positive Selection

The triglyceride compound 1 (Example 1) was conjugated to DNA as follows: Biotin labelled amino-modified oligonucleotide 5′-/5AmMC6//iBio/-(SEQ ID NO: 7)-3′ was synthesized at 10 μmole scale by Integrated DNA Technologies, Inc. (Coralville, Iowa USA) and purified by HPLC. The oligonucleotide is dissolved in water at a concentration of 4 mM to which 20 equivalents of CTAC (40 mM) are added. Upon addition of CTAC, a white precipitate immediately appeared and was pelleted by centrifugation. The supernatant was removed and the CTAC DNA salt dissolved into minimal THF. The THF was then removed under vacuum and the CTAC DNA salt dissolved into methanol at a concentration of 15 mM. Separately, 3 equivalents of bifunctional linker GMBS (N-[γ-maleimidobutyryloxy]succinimide ester, Fisher Scientific PI-22309) and triglyceride compound 1 (LSS1) were dissolved into THF at 20 mM followed by the addition of 10 equivalents of DiPEA (diisopropylethylamine). The reaction proceeded at room temperature for 20 minutes and then was transferred directly to the CTAC DNA salt dissolved in MeOH. The reaction mixture was then left at room temperature overnight and monitored by LCMS. Upon completion, the solvent was removed under vacuum and the resulting product purified using semi-preparative HPLC.

Several additional substrates were synthesized in a similar manner (See, Table 1).

TABLE 1
Substrates Synthesized for Positive Selection

| Oligo modifier | | | |
|---|---|---|---|
| combinations | Bifunctional Linker | Calc Mass | Actual Mass |
| [5AmMC6][iSp18][iBioTEG] | NHS-PEG₆-Maleimide | 9288.3 | 9288.6 |
| | SMCC | 9021.0 | 9021.0 |
| | GMBS | 8966.9 | 8967.2 |
| | MBS | 9000.9 | 9001.1 |
| [5AmMC6][iBioTEG] | NHS-PEG₆-Maleimide | 9632.6 | 9633.0 |
| [5AmMC6][iSp18][iBio] | NHS-PEG₆-Maleimide | 9422.4 | 9422.6 |
| | GMBS | 9101.0 | 9101.2 |
| [5AmMC6][iBio] | NHS-PEG₆-Maleimide | 9766.7 | 9767.0 |
| | MBS | 9479.3 | 9479.5 |
| | GMBS | 9445.3 | 9445.7 |
| | SMCC | 9499.4 | 9499.4 |

Oligo modifiers

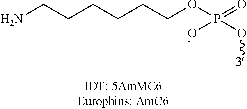

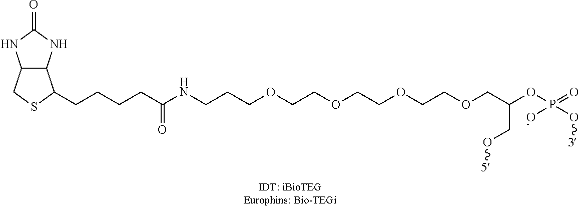

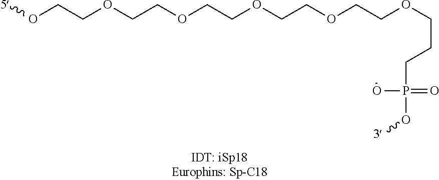

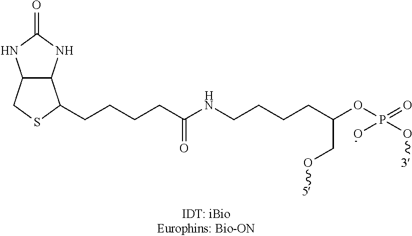

### NHS-Ester Maleimide Bifunctional Linkers:

NHS-PEG2-Maleimide (Thermo Fisher 22102)
NHS-PEG6-Maleimide (Thermo Fisher 22105)
SMCC (succinimidyl 4-(N-maleimidomethyl)cyclohexane-1-carboxylate) (Thermo Fisher 22360)
MBS (m-maleimidobenzoyl-N-hydroxysuccinimide ester) (Thermo Fisher 22311)

The resulting conjugated product was used in a PCR with template A (SEQ ID NO: 1, containing the coding sequence of SEQ ID NO: 2, and encodes for the polypeptide of SEQ ID NO: 3) as follows: A 50 μL aq. reaction was assembled containing 0.5 μM Primer 1 (5′-LSS1-(SEQ ID NO: 7)-3′), 0.5 μM Primer 2 (SEQ ID NO: 8), 500 pg of template A, and 25 μL Q5® High-Fidelity 2× Master Mix (New England Biolabs, Ipswich, Mass. USA). The reaction was activated at 98° C. for 30 s and then thermal cycled 28 times (98° C. for 5 s, 65° C. for 20 s, 72° C. for 20 s) followed by a final extension at 72° C. The resulting 903 bp biotinylated triglyceride amplicon was purified from residual PCR components using the Agencourt AMPure XP system (Beckman Coulter, Inc., Indianapolis, Ind. USA) according to the manufacture's protocol.

### Example 11

### Separation of Hydrolyzed and Non-Hydrolyzed Synthetic Compounds Using Affinity Capture For Positive Selection

The triglyceride amplicon of Example 10 was either hydrolyzed with purified Thermomyces lanuginosus lipase or not hydrolyzed and then selectively retained by affinity capture as follows: A fraction of the triglyceride amplicon from Example 10 was hydrolyzed with purified Thermomyces lanuginosus lipase for 0.5 to 24 hr at 37° C. in 50 mM HEPES, pH 7.6 then diluted five-fold into 100 μL of 1× Binding Buffer (5 mM Tris, pH 8.0, 0.5 mM EDTA, 1 M NaCl, 1% Tween 20). A non-hydrolyzed fraction of the triglyceride amplicon from Example X was diluted to a comparable concentration in 100 μL of 1× Binding Buffer. For each hydrolyzed and non-hydrolyzed sample, 10 μL Dynabeads® MyOne™ Streptavidin C1 magnetic beads (Thermo Fisher Scientific, Inc.) were washed twice, first in a 100 μL then in a 150 μL volume of 1× Binding Buffer using a Kingfisher automated magnetic particle processor (Thermo Scientific, Inc.). The washed beads were each resuspended in the 100 μL hydrolyzed and non-hydrolyzed sample. The amplicons were collected on the beads for 30 min at RT. The biotin tag in non-hydrolyzed synthetic compounds is sequestered by hydrophobic interactions with the triglyceride substrate under certain buffer conditions (preferentially 1 M NaCl, 1% Tween 20 for 30 min at RT). Hydrolyzed synthetic compounds reveal the affinity tag and specifically bind to the streptavidin beads whereas non-hydrolyzed synthetic compounds bind with little to no affinity. The beads are then washed 3 times in 150 μL 1× binding buffer, 1 time in 150 μL 0.1× binding buffer, then released in 100 μL of IDTE+0.01% Tween 20.

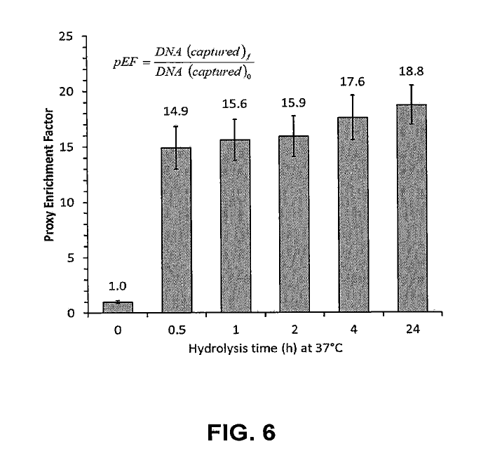

The concentration of released DNA molecules was measured by qPCR as follows: a 15 μL aq. reaction was assembled containing 0.5 μM Primer 1 (5′-GAGGT CTCGC AGGAT CTGT-3′; SEQ ID NO: 9) and 0.5 μM Primer 2 (5′-GCTGG GGCAT CATTG TTT-3′; SEQ ID NO: 10), 5 μL of diluted bead-bound DNA, and 7.5 μL SsoAdvanced™ Universal SYBR® Green Supermix (Bio-Rad, Hercules, Calif. USA). The reactions were activated at 95° C. for 30 s, thermal cycled 45 times (95° C. for 5 s, 60° C. for 5 s), followed by a melting curve measurement (95° C. for 5 s, 65° C. for 1 m, continuous increase to 95° C.), and finally cooled to 48° C. for 2 m. Thermal cycling and measurement of the SYBR® Green signal for the qPCR was performed using a LightCycler® 480 II (Roche, Basel, Switzerland). Enrichment in FIG. 6 is presented as the Proxy Enrichment Factor, which is the quotient of the final DNA captured and the starting DNA captured.

Although the foregoing has been described in some detail by way of illustration and example for the purposes of clarity of understanding, it is apparent to those skilled in the art that any equivalent aspect or modification may be practiced. Therefore, the description and examples should not be construed as limiting the scope of the invention.

1. A method of selecting for a polypeptide having lipase activity, the method comprising:
(i) suspending a plurality of synthetic compounds in an aqueous phase, wherein the synthetic compounds individually comprise:
(a) a polynucleotide encoding for a polypeptide, and
(b) a lipase substrate linked to said polynucleotide; and
wherein the aqueous phase comprises components for expression of the polypeptide;
(ii) forming a water-in-oil emulsion with the aqueous phase, wherein the synthetic compounds are compartmentalized in aqueous droplets of the emulsion;
(iii) expressing the polypeptides within the aqueous droplets of the emulsion, wherein a polypeptide with lipase activity in an aqueous droplet hydrolyzes one or more synthetic compounds in that droplet; and
(iv) separating the synthetic compounds to recover hydrolyzed and/or non-hydrolyzed synthetic compounds.

2. The method of claim 1, wherein the lipase substrate is a triglyceride.

3. The method of claim 2, wherein the polynucleotide encoding for a polypeptide is linked at the 2 position of the triglyceride.

4. The method of claim 1, wherein the synthetic compounds further comprise a selectable marker.

5. The method of claim 4, wherein the selectable marker is linked to the lipase substrate.

6. The method of claim 4, wherein an expressed polypeptide having lipase activity in an aqueous droplet cleaves the selectable marker from one or more of the synthetic compounds in that droplet, thereby allowing selective removal of the non-hydrolyzed synthetic compounds of step (iv).

7. The method of claim 4, wherein the selectable marker is linked to the polynucleotide encoding for a polypeptide.

8. The method of claim 4, wherein the selectable marker is sequestered by the non-hydrolyzed synthetic compounds, thereby allowing selective removal of the hydrolyzed synthetic compounds of step (iv).

9. The method of claim 4, wherein the selectable marker is an affinity tag.

10. The method of claim 9, wherein the hydrolyzed synthetic compounds of step (iv) are separated from the non-hydrolyzed synthetic compounds with streptavidin.

11. A synthetic compound comprising:
(a) a polynucleotide encoding for a polypeptide; and
(b) a lipase substrate linked to said polynucleotide.

12. The synthetic compound of claim 11, wherein the lipase substrate is a triglyceride.

13. The synthetic compound of claim 12, wherein the polynucleotide encoding for a polypeptide is linked at the 2 position of the triglyceride.

14. The synthetic compound of claim 11, wherein the polypeptide has lipase activity.

15. The synthetic compound of claim 11, further comprising a selectable marker.

16. The synthetic compound of claim 15, wherein the selectable marker is linked to the lipase substrate.

17. The synthetic compound of claim 15, wherein the selectable marker is linked to the polynucleotide encoding for a polypeptide.

18. The synthetic compound of claim 11, wherein the selectable marker is an affinity tag.

19. A method of making the synthetic compound of claim 11, comprising:
(i) linking a lipase substrate to a polynucleotide encoding for a polypeptide; and
(ii) recovering the synthetic compound.

20. A polynucleotide library comprising a plurality of different synthetic compounds according to claim 11.

21. A water-in-oil emulsion comprising the polynucleotide library of claim 20, wherein the synthetic compounds are compartmentalized in aqueous droplets of the emulsion.

22. A method of making the emulsion of claim 21, comprising:
(i) suspending the plurality of synthetic compounds in the aqueous phase; and
(ii) mixing the suspension of (i) with an oil.
